(19) United States
(12) Patent Application Publication
(10) Pub. No.: US 2025/0136682 A1
(43) Pub. Date: May 1, 2025

(54) ANTI-SIGLEC-3 ANTIBODIES, PHARMACEUTICAL COMPOSITIONS COMPRISING THE SAME, AND USES THEREOF

(71) Applicant: Academia Sinica, Taipei (TW)

(72) Inventors: Shie-Liang HSIEH, Taipei City (TW); Pei-Shan SUNG, Taoyuan City (TW); An-Suei YANG, Taipei City (TW); Chung-Ming YU, Taipei City (TW)

(21) Appl. No.: 18/837,195

(22) PCT Filed: Apr. 1, 2023
(86) PCT No.: PCT/US2023/065249

§ 371 Date: Aug. 9, 2024

Related U.S. Application Data
(60) Provisional application No. 63/327,809, filed on Apr. 6, 2022.

(51) Int. Cl. C07K 16/28; A61K 31/519; A61K 39/00; A61K 45/06; A61P 25/28; A61P 31/20; A61P 37/06

(52) U.S. Cl. CPC C07K 16/2803; A61K 31/519; A61K 45/06; A61P 25/28; A61P 31/20; A61P 37/06; A61K 2039/505

(57) ABSTRACT

Disclosed herein is generally directed to antibodies against the Siglec-3, and pharmaceutical compositions that comprise the antibodies. According to some embodiments of the present disclosure, the present anti-Siglec-3 antibodies may suppress the over activation of Siglec-3, thereby reversing the immunosuppression effects resulted therefrom. As such, the present antibodies may treat diseases associated with the over activation of Siglec-3, such as hepatitis B virus (HBV) infection, neurodegenerative diseases, autoimmune diseases, or cancers. Accordingly, the present disclosure also includes methods for the treatment and/or prophylaxis of the above diseases.

### CROSS-REFERENCE OF RELATED APPLICATION

This application claims priority to US. Application No. 63/327,809, filed on Apr. 6, 2022. The content of which application is incorporated herein by reference in its entirety.

### SEQUENCE LISTING XML

The present application is being filed along with a Sequence Listing XML in electronic format. The Sequence Listing XML is provided as an XML file entitled “AJ23006_SeqList_20230327.xml,” created Mar. 27, 2023, which is 164 Kb in size. The information in the electronic format of the Sequence Listing XML is incorporated herein by reference in its entirety.

### BACKGROUND OF THE INVENTION

### 1. Field of the Invention

The present disclosure in general relates to anti-Siglec-3 antibodies and therapeutic uses of such antibodies in treating hepatitis B virus (HBV) infection, neurodegenerative diseases, autoimmune diseases, and cancers.

### 2. Description of Related Art

Siglec-3 (also named CD33) is an I-type, immunoglobulin (Ig)-like, transmembrane receptor that is prominently expressed on innate immune cells (such as monocytes, basophils, CD34⁺ cells, dendritic cells, macrophages, mast cells, neutrophils), hematopoietic cells (such as myeloid progenitors), as well as cells in the nervous system (such as microglia). Siglec-3 is a member of the Siglec protein family, and has been reported to exert its function as an inhibitory receptor and a negative regulator of immune functions, which is achieved by binding sialic acid-containing ligands via its amino-terminal extracellular Ig-like domains and modulates cellular responses via its cytosolic signaling motifs, immunoreceptor tyrosine-based inhibitory motif (ITIM), leading to recruitment of the tyrosine phosphatases SHP1 and SHP2. As such, over activation of Siglec-3 may potentially lead to diseases resulted from imbalance of the hosts' immune system. To date, the Siglec protein family is known to be associated with many human diseases, including immune diseases (e.g., systemic lupus erythematosus (SLE), rheumatoid arthritis, asthma, allergy, graft-versus-host disease), susceptibility to infection (such as HBV infection; or infection-related conditions, for example, sepsis, eosinophilia), multiple types of cancer (including lymphoma, leukemia, acute myeloid leukemia (AML), and hepatocellular carcinoma), neurodegenerative disorders (in particular, Alzheimer's disease), chronic obstructive pulmonary disease, and osteoporosis.

It has been suggested that reduction of expression or functionality of Siglec-3 may be beneficial in Alzheimer's disease and cancer, and one of the feasible means to achieve this is with the aid of employment of anti-Siglec-3 antibodies. Nonetheless, no data has been reported on the ability of any current anti-Siglec-3 antibodies to downregulate Siglec-3, or block Siglec-3 ligand/receptor interactions, in physiologically relevant immune cells.

In view of the foregoing, there exists in the related art a need for therapeutic antibodies for treating one or more diseases, disorders, and conditions associated with undesired Siglec-3 activity.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a recombinant antibody against the therapeutic target Siglec-3. The recombinant antibody comprises a light chain variable (VL) region and a heavy chain variable (VH) region, in which the VL region comprises a first complementarity determining region (CDR-L1), a second complementarity determining region (CDR-L2), and a third complementarity determining region (CDR-L3); and the VH region comprises a first complementarity determining region (CDR-H1), a second complementarity determining region (CDR-H2), and a third complementarity determining region (CDR-H3), wherein
the CDR-L1 is selected from the group consisting of SEQ ID NOs: 1 and 57;
the CDR-L2 is selected from the group consisting of SEQ ID NOs: 2 and 58;
the CDR-L3 is selected from the group consisting of SEQ ID NOs: 3 and 59;
the CDR-H1 is selected from the group consisting of SEQ ID NOs: 5, 9, 13, 17, 21, 25, 29, 33, 37, 41, 45, 49, 53, and 61;
the CDR-H2 is selected from the group consisting of SEQ ID NOs: 6, 10, 14, 18, 22, 26, 30, 34, 38, 42, 46, 50, 54, and 62; and
the CDR-H3 is selected from the group consisting of SEQ ID NOs: 7, 11, 15, 19, 23, 27, 31, 35, 39, 43, 47, 51, 55, and 63.

According to the embodiments of the present disclosure, the recombinant antibody has
the CDR-L1, the CDR-L2, the CDR-L3, the CDR-H1, the CDR-H2, and the CDR-H3 respectively comprising the amino acid sequences of SEQ ID NOs: 1, 2, 3, 5, 6, and 7;
the CDR-L1, the CDR-L2, the CDR-L3, the CDR-H1, the CDR-H2, and the CDR-H3 respectively comprising the amino acid sequences of SEQ ID NOs: 1, 2, 3, 9, 10, and 11;
the CDR-L1, the CDR-L2, the CDR-L3, the CDR-H1, the CDR-H2, and the CDR-H3 respectively comprising the amino acid sequences of SEQ ID NOs: 1, 2, 3, 13, 14, and 15;
the CDR-L1, the CDR-L2, the CDR-L3, the CDR-H1, the CDR-H2, and the CDR-H3 respectively comprising the amino acid sequences of SEQ ID NOs: 1, 2, 3, 17, 18, and 19;
the CDR-L1, the CDR-L2, the CDR-L3, the CDR-H1, the CDR-H2, and the CDR-H3 respectively comprising the amino acid sequences of SEQ ID NOs: 1, 2, 3, 21, 22, and 23;
the CDR-L1, the CDR-L2, the CDR-L3, the CDR-H1, the CDR-H2, and the CDR-H3 respectively comprising the amino acid sequences of SEQ ID NOs: 1, 2, 3, 25, 26, and 27;
the CDR-L1, the CDR-L2, the CDR-L3, the CDR-H1, the CDR-H2, and the CDR-H3 respectively comprising the amino acid sequences of SEQ ID Nos: 1, 2, 3, 29, 30, and 31;
the CDR-L1, the CDR-L2, the CDR-L3, the CDR-H1, the CDR-H2, and the CDR-H3 respectively comprising the amino acid sequences of SEQ ID NOs: 1, 2, 3, 33, 34, and 35;
the CDR-L1, the CDR-L2, the CDR-L3, the CDR-H1, the CDR-H2, and the CDR-H3 respectively comprising the amino acid sequences of SEQ ID NOs: 1, 2, 3, 37, 38, and 39;
the CDR-L1, the CDR-L2, the CDR-L3, the CDR-H1, the CDR-H2, and the CDR-H3 respectively comprising the amino acid sequences of SEQ ID Nos: 1, 2, 3, 41, 42, and 43;
the CDR-L1, the CDR-L2, the CDR-L3, the CDR-H1, the CDR-H2, and the CDR-H3 respectively comprising the amino acid sequences of SEQ ID NOs: 1, 2, 3, 45, 46, and 47;
the CDR-L1, the CDR-L2, the CDR-L3, the CDR-H1, the CDR-H2, and the CDR-H3 respectively comprising the amino acid sequences of SEQ ID NOs: 1, 2, 3, 49, 50, and 51;
the CDR-L1, the CDR-L2, the CDR-L3, the CDR-H1, the CDR-H2, and the CDR-H3 respectively comprising the amino acid sequences of SEQ ID NOs: 1, 2, 3, 53, 54, and 55; or
the CDR-L1, the CDR-L2, the CDR-L3, the CDR-H1, the CDR-H2, and the CDR-H3 respectively comprising the amino acid sequences of SEQ ID NOs: 57, 58, 59, 61, 62, and 63.

According to the embodiments of the present disclosure, the recombinant antibody has
the VL and the VH regions respectively comprising an amino acid sequence at least 85% identical to SEQ ID NOs: 4 and 8;
the VL and the VH regions respectively comprising an amino acid sequence at least 85% identical to SEQ ID NOs: 4 and 12;
the VL and the VH regions respectively comprising an amino acid sequence at least 85% identical to SEQ ID NOs: 4 and 16;
the VL and the VH regions respectively comprising an amino acid sequence at least 85% identical to SEQ ID NOs: 4 and 20;
the VL and the VH regions respectively comprising an amino acid sequence at least 85% identical to SEQ ID NOs: 4 and 24;
the VL and the VH regions respectively comprising an amino acid sequence at least 85% identical to SEQ ID NOs: 4 and 28;
the VL and the VH regions respectively comprising an amino acid sequence at least 85% identical to SEQ ID NOs: 4 and 32;
the VL and the VH regions respectively comprising an amino acid sequence at least 85% identical to SEQ ID NOs: 4 and 36;
the VL and the VH regions respectively comprising an amino acid sequence at least 85% identical to SEQ ID NOs: 4 and 40;
the VL and the VH regions respectively comprising an amino acid sequence at least 85% identical to SEQ ID NOs: 4 and 44;
the VL and the VH regions respectively comprising an amino acid sequence at least 85% identical to SEQ ID NOs: 4 and 48;
the VL and the VH regions respectively comprising an amino acid sequence at least 85% identical to SEQ ID NOs: 4 and 52;
the VL and the VH regions respectively comprising an amino acid sequence at least 85% identical to SEQ ID NOs: 4 and 56; or
the VL and the VH regions respectively comprising an amino acid sequence at least 85% identical to SEQ ID NOs: 60 and 64.

Preferably, the recombinant antibody has
the VL and the VH regions respectively comprising the amino acid sequences of SEQ ID NOs: 4 and 8;
the VL and the VH regions respectively comprising the amino acid sequences of SEQ ID NOs: 4 and 12;
the VL and the VH regions respectively comprising the amino acid sequences of SEQ ID NOs: 4 and 16;
the VL and the VH regions respectively comprising the amino acid sequences of SEQ ID NOs: 4 and 20;
the VL and the VH regions respectively comprising the amino acid sequences of SEQ ID NOs: 4 and 24;
the VL and the VH regions respectively comprising the amino acid sequences of SEQ ID NOs: 4 and 28;
the VL and the VH regions respectively comprising the amino acid sequences of SEQ ID NOs: 4 and 32;
the VL and the VH regions respectively comprising the amino acid sequences of SEQ ID NOs: 4 and 36;
the VL and the VH regions respectively comprising the amino acid sequences of SEQ ID NOs: 4 and 40;
the VL and the VH regions respectively comprising the amino acid sequences of SEQ ID NOs: 4 and 44;
the VL and the VH regions respectively comprising the amino acid sequences of SEQ ID NOs: 4 and 48;
the VL and the VH regions respectively comprising the amino acid sequences of SEQ ID NOs: 4 and 52;
the VL and the VH regions respectively comprising the amino acid sequences of SEQ ID NOs: 4 and 56; or
the VL and the VH regions respectively comprising the amino acid sequences of SEQ ID NOs: 60 and 64.

Accordingly, another aspect of the present disclosure aims at providing a pharmaceutical composition for preventing and/or treating HBV infection, a neurodegenerative disease, an autoimmune disease, or a cancer. The pharmaceutical composition comprises an effective amount of any of the recombinant antibody as set forth above, and a pharmaceutically acceptable excipient.

The recombinant antibody of this invention is present at a level of about 0.1% to 99% by weight, based on the total weight of the pharmaceutical composition. In some embodiments, the recombinant antibody of this invention is present at a level of at least 1% by weight, based on the total weight of the pharmaceutical composition. In certain embodiments, the recombinant antibody of this invention is present at a level of at least 5% by weight, based on the total weight of the pharmaceutical composition. In still other embodiments, the recombinant antibody of this invention is present at a level of at least 10% by weight, based on the total weight of the pharmaceutical composition. In still yet other embodiments, the recombinant antibody of this invention is present at a level of at least 25% by weight, based on the total weight of the pharmaceutical composition.

In yet another aspect of the present disclosure, a method for preventing and/or treating HBV infection, a neurodegenerative disease, an autoimmune disease, or a cancer in a subject is provided. The method comprises the step of administering to the subject an effective amount of the present recombinant antibody, or the present pharmaceutical composition as described above.

According to some embodiments of the present disclosure, the present method is used to treat a subject having HBV infection, and may further comprise the step of administering to the subject an effective amount of an antiviral agent prior to, in conjunction with, or subsequent to administering to the subject the present recombinant antibody, or the present pharmaceutical composition. Said antiviral agent includes, but is not limited to, acyclovir, adefovir, brincidofovir, brivudine, cidofovir, clevudine, cytarabine, docosanol, edoxudine, entecavir, famciclovir, filociclovir, fomivirsen, foscarnet, ganciclovir, idoxuridine, imiquimod, interferon-α, lamivudine, lobucavir, maribavir, methisazone, moroxydine, penciclovir, peginterferon-α, podophyllotoxin, ribavirin, rifampicin, resiquimod, sorivudine, taribavirin, tecovirimat, telbivudine, tenofovir, thymosin-α, trifluridine, tromantadine, valaciclovir, valganciclovir, vesatolimod (GS-9620), vidarabine, viread, and a combination thereof. In one working example, the antiviral agent is vesatolimod (GS-9620).

According to some embodiments of the present disclosure, the present method is used to treat a subject having the neurodegenerative disease, which is any one of Alzheimer's disease, Batten disease, Parkinson's disease, dementia with Lewy bodies, multiple system atrophy, bovine spongiform encephalopathy, Creutzfeldt-Jakob disease, Huntington's disease, amyotrophic lateral sclerosis, ataxia telangiectasia, multiple sclerosis, spinocerebellar atrophy, HIV-associated neurocognitive disorders, Pick's disease, Krabbe's disease, spinal and bulbar muscular atrophy, primary lateral sclerosis, Cockayne syndrome, spinal muscular atrophy, tabes dorsalis, progressive supranuclear palsy, or Pelizaeus-Merzbacher disease. In one preferred embodiment, the neurodegenerative disease treatable by the present method is Alzheimer's disease.

Further, in the case when the subject has the neurodegenerative disease, the present method may further comprise the step of administering to the subject an effective amount of a neurodegenerative disease treatment agent prior to, in conjunction with, or subsequent to administering to the subject the present recombinant antibody, or the present pharmaceutical composition. Said neurodegenerative disease treatment agent that may be used in combination with the present recombinant antibody or the present pharmaceutical composition includes, but is not limited to, acyclovir, aducanumab, amantadine, apomorphine, baclofen, carbidopa, carbidopa, dantrolene, donepezil, entacapone, foscarnet, galantamine, levodopa, memantine, penciclovir, pramipexole, rasagiline, riluzole, rivastigmine, ropinirole, selegiline, tacrine, tetrabenazine, tizanidine, or tolcapone.

According to some embodiments of the present disclosure, the present method is used to treat a subject having the autoimmune disease. Such autoimmune disease may be any one of acute disseminated encephalomyelitis, Addison's disease, alopecia areata, antiphospholipid syndrome, antisynthetase syndrome, asthma, autoimmune angioedema, autoimmune hepatitis, autoimmune pancreatitis, autoimmune polyendocrine syndrome type 1-3, autoimmune progesterone dermatitis, autoimmune thyroiditis, autoimmune urticaria, bullous pemphigoid, chronic hives, cicatricial pemphigoid, coeliac disease, Crohn's disease, dermatitis herpetiformis, dermatomyositis, diabetes mellitus type 1, discoid lupus erythematosus, endometriosis, epidermolysis bullosa acquisita, esophageal achalasia, erythema nodosum, familial mediterranean fever, gestational pemphigoid, Graves' disease, gout, hidradenitis suppurativa, IgA vasculitis, inflammatory bowel disease, interstitial cystitis, lichen planus, lichen sclerosus, lupus nephritis, morphea, Mucha-Habermann disease, Muckle-Wells syndrome, myocarditis, myasthenia gravis, pemphigus vulgaris, pityriasis lichenoides et varioliformis acuta, postmyocardial infarction syndrome, primary biliary cholangitis, primary sclerosing cholangitis, psoriasis, rheumatoid arthritis, Sjögren syndrome, subacute bacterial endocarditis, systemic lupus erythematosus (SLE), systemic scleroderma, ulcerative colitis, or vitiligo. In one preferred embodiment, the autoimmune disease treatable by the present method is SLE.

Alternatively or in addition, in the case when the subject has the autoimmune disease, the present method may further comprise the step of administering to the subject an effective amount of an autoimmune disease treatment agent prior to, in conjunction with, or subsequent to administering to the subject the present recombinant antibody, or the present pharmaceutical composition. Said autoimmune disease treatment agent suitable for use in combination with the present recombinant antibody or the present pharmaceutical composition may be abatacept, acitretin, adalimumab, alefacept, anakinra, anthralin, apremilast, azathioprine, baricitinib, belimumab, benralizumab, betamethasone, bimekizumab, brazikumab, brodalumab, calcipotriene, calcipotriol, calcitriol, canakinumab, certolizumab, clobetasol, coal tar, colchicine, cyclosporine, dapsone, dithranol, dexamethasone, dupilumab, eculizumab, etanercept, fluocinolone, golimumab, guselkumab, hydroxychloroquine, hydrocortisone, infliximab, ixekizumab, lenercept, mepolizumab, methotrexate, methylprednisolone, mirikizumab, mycophenolate mofetil, omalizumab, prednisone, perakizumab, pimecrolimus, remtolumab, reslizumab, rilonacept, risankizumab, rituximab, sarilumab, secukinumab, sulfasalazine, tacrolimus, tazarotene, tildrakizumab, tocilizumab, tofacitinib, tretinoin, upadacitinib, ustekinumab, vedolizumab, or vunakizumab.

According to some embodiments of the present disclosure, the present method is used to treat a subject having the cancer. Exemplary cancer that is treatable with the present method may be any one of bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer.

Optionally, in the case when the subject has the cancer, the present method may further comprise the step of administering to the subject an effective amount of an anti-cancer drug prior to, in conjunction with, or subsequent to administering to the subject the present recombinant antibody, or the present pharmaceutical composition. Said an anti-cancer drug that may be used in the present method in combination with the present recombinant antibody or the present pharmaceutical composition may be actinomycin D, altretamine, aminoglutethimide, amsacrin, anastrozole, anthracycline, asparaginase, bendamustine, bexarotene, bleomycin, bortezomib, buserelin, busulfan, camptothecin, capecitabine, carboplatin, carmustine, chlorambucil, chlormethine, cisplatin, cladribine, clofarabine, clomifene, curcumin, cyclophosphamide, cytarabine, cytosinarabinoside, dacarbazine, dactinomycin, daunorubicin, dexamethasone, docetaxel, doxorubicin, epirubicin, estramustine, estrone, estradiol, estriol, etoposide, etoposid, exemestane, fludarabine, fluorouracil, formestane, foxuridine, gemcitabine, glucocorticoid, goserelin, hycamtin, hydroxy urea, idarubicin, ifosfamid, imatinib, indirubin, irinotecan, ixabepilone, letrozole, leuprorelin, lomustine, mechlorethamine, melphalan, mercaptopurine, methotrexate, miltefosin, mitomycine, mitoxantrone, nintedanib, nimustine, oxaliplatin, paclitaxel, pentostatin, plicamycin, prednisone, procarbazine, progesterone, raloxifene, streptozocin, tamoxifen, temozolomide, teniposide, testolactone, testosterone, thiotepa, thioguanine, topotecan, treosulfan, tretinoin, triptorelin, trofosfamide, vinblastine, vincristine, vindesine, or vinorelbine.

The subject treatable bythe present method is a mammal, for example, a human, a mouse, a rat, a guinea pig, a hamster, a monkey, a swine, a dog, a cat, a horse, a sheep, a goat, a cow, and a rabbit. Preferably, the subject is a human.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIGS. 1A-1D are the results depicting the binding affinity of the antibody 4H3-3A toward Siglec-3. FIGS. 1A-1C are the titration curves of the antibody 4H3-3A on human primary cells, including monocyte-derived dendritic cells (moDCs) (FIG. 1A) and monocytes (FIG. 1B), which endogenously expressed Siglec-3, as well as on human embryonic kidney cells-293T (HEK-293T) cells that expressed exogenous human or mouse Siglec-3 (FIG. 1C), which were measured by flow cytometry according to Example 2 of the present disclosure, depicting the binding affinity of the antibody 4H3-3A toward either endogenous or exogenous Siglec-3 expressed in the cells. FIG. 1D is the result depicting the binding affinity of the antibody 4H3-3A toward human Siglec-3 using the Biacore analysis. MFI: mean fluorescence intensity.

FIGS. 2A-2F illustrate the effect of the antibody 4H3-3A on the expression of the surface markers in CD14⁺ peripheral blood mononuclear cells (PBMCs) derived from the chronic hepatitis B (CHB) patients (as HBV carriers) or the healthy donors, as measured by flow cytometry, in which FIGS. 2A-2F show the expression results of Siglec-3 (FIG. 2A), CD80 (FIG. 2B), CD86 (FIG. 2C), PD-L1 (FIG. 2D), MHC-I (FIG. 2E), and MHC-II (FIG. 2F), after treatments with the antibodies 4H3-3A and 10C8, either alone or in combination with GS-9620, respectively. All data were expressed as means±standard deviation (S.D.); *: p<0.05, **: p<0.01, ***: p<0.001.

FIGS. 3A-3D depict the ability of the antibody 4H3-3A to modulate the immunity against HBV infection. FIGS. 3A-3C show the cytokine secretions by the PBMCs derived from the healthy donors (upper panels) or the CHB patients (lower panels), as measured by enzyme-linked immunosorbent assay (ELISA), in which FIGS. 3A-3C depict the secretion levels of IFN-α (FIG. 3A), TNF-α (FIG. 3B), and IL-6 (FIG. 3C), after treatments with the antibodies 4H3-3A and 10C8, either alone or in combination with GS-9620, respectively. FIG. 3D displays the induction of anti-HBsAg antibodies by the PBMCs derived from the CHB patients after treatment with the antibody 4H3-3A, as measured by enzyme-linked immunosorbent spot (ELISpot) (left panel) or ELISA (right panel), following Example 2 of the present disclosure. All data were expressed as means±S.D.; *: p<0.05, **: p<0.01, ***: p<0.001. N.D.: non-detectable.

FIGS. 4A-4C illustrate the ability of the antibody 4H3-3A to enhance the phargocytosis of disease-associated proteins by the iPS-derived human microglia for the treatment of Alzheimer's disease. FIGS. 4A-4B show the percentage (%) (FIG. 4A) and intensity (FIG. 4B) of β-amyloid peptide 1-42 (Aβ₁₋₄₂) phagocytosis by the iPS-derived human microglia, and FIG. 4C displays the intensity of Tau phagocytosis by the iPS-derived human microglia, after treated with the indicated antibodies. MFI: mean fluorescence intensity.

FIG. 5 demonstrates the inhibitory effect of the antibody 4H3-3A on the formation of neutrophil extracellular traps (NETs) from the extracellular vesicles (EVs) derived from the SLE patients, so as to treat SLE.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of molecular biology, microbiology, recombinant DNA, and immunology, which are within the skill of the art. Such techniques are explained fully in the literature.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “antibody” is used in the broadest sense and specifically covers monoclonal antibodies (including full length monoclonal antibodies), polyclonal antibodies, multi-specific antibodies (e.g., bi-specific antibodies), and antibody fragments so long as they exhibit the desired biological activity.

“Antibody fragments” comprise a portion of a full-length antibody, wherein the portion retains at least one, and as many as most or all, of the functions normally associated with that portion when present in the full-length antibody, generally the antigen-binding site or variable region thereof. Accordingly, an antibody fragment comprises an antigen binding site of the full-length antibody and thus retains the ability to bind antigen. Alternatively, an antibody fragment may comprise the Fc region and retains at least one of the biological functions normally associated with the Fc region when present in the full-length antibody, such as FcRn binding, antibody half-life modulation, ADCC function and complement binding. The antibody fragment in the present invention may exist in a variety of forms including, without limitation, an antigen-binding fragment (Fab), Fab′, F(ab′)₂, and a variable fragment (Fv); a diabody; a linear antibody; a single-chain antibody molecule (e.g., a single-chain fragment variable (scFv)); a single domain antibody (e.g., a variable heavy domain (V_{H}H)); and a multi-specific antibody formed from an antibody fragment.

The “variable region” or “variable domain” of an antibody refers to the amino-terminal domains of heavy or light chain of the antibody. These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

The term “complementarity determining region” (CDR) used herein refers to the hypervariable region of an antibody molecule that forms a surface complementary to the 3-dimensional surface of a bound antigen. Proceeding from N-terminus to C-terminus, each of the antibody heavy and light chains comprises three CDRs (CDR 1, CDR 2, and CDR3). A HLA-DR antigen-binding site, therefore, includes a total of six CDRs, which includes three CDRs from the variable region of a heavy chain and three CDRs from the variable region of a light chain.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

As discussed herein, minor variations in the amino acid sequences of antibodies are contemplated as being encompassed by the presently disclosed and claimed inventive concept(s), providing that the variations in the amino acid sequences maintain at least 85% sequence identity, such as at least 85%, 86%, 87%, 88%, 89%, 90%, 91%, 92%, 93%, 94%, 95%, 96%, 97%, 98% and 99% sequence identity. Antibodies of the present disclosure may be modified specifically to alter a feature of the peptide unrelated to its physiological activity. For example, certain amino acids can be changed and/or deleted without affecting the physiological activity of the antibody in this study (i.e., its ability to bind Siglec-3). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of antibodies can be readily prepared by those of ordinary skill in the art. Preferred amino- and carboxyl-termini of fragments or analogs occur near boundaries of functional domains. In one example, one amino acid residue (e.g., valine) of the present antibody is conservatively replaced (e.g., by leucine). In other examples, two amino acid residues of the present antibody are conservatively replaced by other suitable amino acid residues, for example, valine (V) and arginine (R) are replaced by the pair of amino acids that includes, but is not limited to, methionine (M) and lysine (K), lysine (K) and proline (P), tryptophan (W) and isoleucine (I), isoleucine (I) and proline (P), asparagine (N) and valine (V), and glutamine (G) and lysine (K).

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100$$

where X is the number of amino acid residues scored as identical matches by the sequence alignment program BLAST in that program's alignment of A and B, and where Y is the total number of amino acid residues in A or B, whichever is shorter.

The phrase “pharmaceutically acceptable excipient” as used herein means a pharmaceutically acceptable material, composition or vehicle, such as a liquid or solid filler, diluent, carrier, solvent or encapsulating material, involved in carrying or transporting the subject agents from one organ, or portion of the body, to another organ, or portion of the body. Each carrier must be “acceptable” in the sense of being compatible with the other ingredients of the formulation. The pharmaceutical formulation contains the recombinant antibody of the invention in combination with one or more pharmaceutically acceptable ingredients. The carrier can be in the form of a solid, semi-solid or liquid diluent. These pharmaceutical preparations are a further object of the invention. For the clinical use of the present method, the pharmaceutical composition of the invention is formulated into formulations suitable for the intended route of administration.

The term “prevent,” “preventing” and “prophylaxis” as used herein are interchangeable, and refers to the prophylactic treatment (e.g., a recombinant antibody, a pharmaceutical composition comprising the recombinant antibody, and/or a method of the present invention) of a subject who is at risk of developing a symptom, a secondary disorder or a condition associated with HBV infection, a neurodegenerative disease, an autoimmune disease, or a cancer, so as to decrease the probability that the subject will develop the symptom, secondary disorder or condition. The term “prevent,” “preventing” or “prophylaxis” does not mean or imply that use of the recombinant antibody, the pharmaceutical composition comprising the recombinant antibody, and/or the method of the present invention will provide a guarantee that the symptom, secondary disorder or condition will never occur, but rather that the recombinant antibody, the pharmaceutical composition comprising the recombinant antibody, and/or the method of the present invention will inhibit the occurrence of the symptom, secondary disorder or condition, and that the incidence and/or frequency of the symptom, secondary disorder or condition will be reduced.

As used herein, the term “treat,” “treating,” and “treatment” are interchangeable, and encompasses partially or completely preventing, ameliorating, mitigating and/or managing a symptom, a secondary disorder or a condition associated with HBV infection, a neurodegenerative disease, an autoimmune disease, or a cancer, with application or administration of the present antibody to a subject, who has a symptom, a secondary disorder or a condition associated with HBV infection, a neurodegenerative disease, an autoimmune disease, or a cancer, with the purpose to partially or completely alleviate, ameliorate, relieve, delay onset of, inhibit progression of, reduce severity of, and/or reduce incidence of one or more symptoms, secondary disorders or features associated with HBV infection, a neurodegenerative disease, an autoimmune disease, or a cancer. Symptoms, secondary disorders, and/or conditions associated with HBV infection, a neurodegenerative disease, an autoimmune disease, or a cancer include, but are not limited to, fatigue, nausea, vomiting, dark urine, joint and muscle pain, loss of appetite, fever, abdominal discomfort, weakness, jaundice, and liver failure. Treatments may be administered to a subject who exhibits only early signs of such symptoms, disorder, and/or condition for the purpose of decreasing the risk of developing the symptoms, secondary disorders, and/or conditions associated with HBV infection, a neurodegenerative disease, an autoimmune disease, or a cancer. Treatments are generally “effective” if one or more symptoms or clinical markers are reduced as that term is defined herein. Alternatively, treatments are “effective” if the progression of a symptom, disorder or condition is reduced or halted.

The term “subject” or “patient” refers to an animal including the human species that is treatable with the recombinant antibody, the pharmaceutical composition, and/or the method of the present disclosure. The term “subject” or “patient” intended to refer to both the male and female gender unless one gender is specifically indicated. Accordingly, the term “subject” or “patient” comprises any mammal which may benefit from treatment of HBV infection, a neurodegenerative disease, an autoimmune disease, or a cancer. In an exemplary example, the subject is a human.

The term “administered,” “administering” or “administration” are used interchangeably herein to refer means either directly administering the recombinant antibody, the pharmaceutical composition comprising the recombinant antibody, and/or the method of the present invention.

The term “an effective amount” as used herein refers to an amount of a component effective, at dosages, and for periods of time necessary, to achieve the desired therapeutically desired results with respect to the treatment of chronic hepatitis B. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/kg). Alternatively, the effective amount can be expressed in the concentration of the active component (e.g., the present antibody), such as molar concentration, mass concentration, volume concentration, molality, mole fraction, mass fraction and mixing ratio. Persons having ordinary skills could calculate the human equivalent dose (HED) for the medicament (such as the present antibody) based on the doses determined from animal models. For example, one mayfollow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

### II. Description of the Invention

The present disclosure is based, at least in part, on the discovery of a series of novel monoclonal antibodies that are specific to the sialic acid-binding immunoglobulin-like lectin-3 (Siglec-3) having the ability to reverse immunosuppression caused by over activation of Siglec-3.

### 1. The Recombinant Antibody

As such, one aspect of the present disclosure provides a recombinant antibody that exhibits binding affinity to Siglec-3 receptor, and the recombinant antibody has the potential to prevent and/or treat HBV infection, a neurodegenerative disease, an autoimmune disease, or a cancer in a subject. According to one preferred embodiment, the recombinant antibody is a monoclonal antibody.

According to the embodiments of the present disclosure, the present antibody is produced by standard immunization methods (i.e., immunizing animals with specific peptides). As would be appreciated, the present antibody may alternatively be produced from phage-displayed scFv libraries. A phage-displayed scFv library is constructed on a phagemid vector, and the method for construction of a phage-displayed scFv library is well known in the art. For selecting a phage-displayed scFv from the phage-displayed scFv library with high binding affinity and specificity to the human Siglec-3, the method comprises the steps of,
(a) incubating the phage-displayed scFv library having a plurality of phage-displayed scFvs with the human Siglec-3 (e.g., the recombinant extracellular domain of the human Siglec-3);
(b) purifying the product of step (a) thereby producing a plurality of phage-displayed scFvs, which were respectively bound to the human Siglec-3 before the purification; and
(c) repeating at least one run of the steps (a) and (b), each time using the product of the step (b) in previous run as the phage-displayed scFv library for incubating with the human Siglec-3, until the phage-displayed scFv exhibiting the highest binding affinity and specificity to the human Siglec-3 is obtained.

In the step (a), the human Siglec-3 (especially for the recombinant extracellular domain of the human Siglec-3) is preferably fused with Fc portion of hIgG1 (Siglec-3.Fc), and the phage-displayed scFv library is preferably a resuspended polyethylene glycol/NaCl-precipitated phage display library.

In the step (b), the product of the step (a) is purified by carrying out conventional purification procedures known in the art, such as an acid-base neutralization method, in which the product of the step (a) is subject to an acid treatment (e.g., treating with an elution buffer of pH 2.2 (e.g., a HCl/glycine solution)) to separate the scFvs from their respective bound antigens. And the resulting produced phage-displayed scFvs are neutralized by adding an alkaline solution, such as a solution having a pH value of 9.0-9.2 (e.g., a 2 M Tris-based solution, pH 9.1).

For the purpose of selecting an scFv exhibiting highest binding affinity and specificity to the human Siglec-3, the steps (a) and (b) are repeated for at least one run, each time using the alkaline-treated phage-displayed scFvs produced in the previous run as the starting phage library for incubating with the human Siglec-3, until the phage-displayed scFv exhibiting the highest binding affinity and specificity to the human Siglec-3 (i.e., the present antibody) is obtained.

Optionally, the alkaline-treated or neutralized phage-displayed scFvs produced in the step (b) may further be amplified in a host cell, for example, in Escherichia coli (E. coli), by infecting the host cell with the phage expressing the neutralized scFvs. Then, the amplified phage-displayed scFvs are incubated with the human Siglec-3, and the steps (a) and (b) are repeated, until the phage-displayed scFv exhibiting the highest binding affinity and specificity to the human Siglec-3 (i.e., the present antibody) is obtained.

The binding affinity and specificity of the resultant scFvs (i.e., the present antibody) may be measured by any suitable method known in the art, which includes, but is not limited to, chemiluminescence immunoassay (CLIA), electrochemiluminescence immunoassay (ECLIA), enzyme-linked immunosorbent assay (ELISA) (including sandwich ELISA), enzyme-linked immunosorbent spot (ELISpot), fluoroimmnoassay (FIA), real-time immunoquantitative PCR (iqPCR), magnetic immunoassay (MIA), radioimmunoassay (RIA), flow cytometry, and Biacore analysis. In one preferred example, the binding affinity and specificity of the scFvs are measured by ELISA. In another preferred example, the binding affinity and specificity of the scFvs are measured by flow cytometry. In another preferred example, the binding affinity and specificity of the scFvs are measured by Biacore analysis.

Once the present antibody in the form of scFv is obtained from the procedures as described above, it may be engineered to change the format of the antibodies (e.g., Fv, Fab, Fab′, F(ab′)2, diabodies, V_{H}H, IgG) via DNA cloning techniques. DNA encoding the scFv may be easily isolated and sequenced by use of conventional procedures, such as using oligonucleotide probes that are capable of binding specifically to genes encoding the heavy and light chains of the scFv. The phages expressing the scFv serve as a preferred source of such DNA. Once isolated, the DNA may be placed into expression vectors, which are then transfected into host cells such as E. coli cells, simian COS cells or Chinese hamster ovary (CHO) cells, or myeloma cells that do not produce immunoglobulin proteins, to synthesize the desired antibodies in the recombinant host cells.

The antibody and the DNA encoding such antibody can then be used to produce chimeric antibodies (e.g., bi-specific antibodies), humanized antibodies, and/or antibody fragments derived thereof. For producing humanized antibodies, variable domains in the heavy and light chains of non-human origin antibody were attached onto the constant regions of human antibodies. The DNA encoding such antibodies was isolated and sequenced, and then used to create humanized constructs. According to some preferred embodiments of the present disclosure, the VH and VL genes of the non-human origin antibody are constructed into a human IgG1 (hIgG1) vector. According to other preferred embodiments of the present disclosure, the VH and VL genes of the non-human origin antibody are constructed into a human IgG4 (hIgG4) vector. The resulting antibody therefore has VH and VL regions derived from the non-human origin antibody, while the constant region genes (i.e., CK (SEQ ID NO: 65), and CH1-H-CH2-CH3 (SEQ ID NO: 66 for IgG1; SEQ ID NO: 67 for IgG4)) are those of human IgG.

According to one specific working example of the present disclosure, the present antibody H3-3A is constructed into a hIgG1 vector, and is termed as the “1H3-3A” antibody. According to another specific working example of the present disclosure, the present antibody H3-3A is constructed into a hIgG4 vector, and is termed as the “4H3-3A” antibody. The 1H3-3A and the 4H3-3A antibodies respectively comprises the full length light chain (LC) and heavy chain (HC) of the amino acid sequences of SEQ ID NOs: 68 and 69 (1H3-3A) and SEQ ID NOs: 68 and 70 (4H3-3A), which are respectively encoded by the nucleotide sequences of SEQ ID NOs: 98 and 99 (1H3-3A) and SEQ ID NOs: 98 and 100 (4H3-3A).

According to another example of the present disclosure, the present antibody H3-7G is constructed into a hIgG1 vector, and is termed as the “1H3-7G” antibody. According to another example of the present disclosure, the present antibody H3-7G is constructed into a hIgG4 vector, and is termed as the “4H3-7G” antibody. The 1H3-7G and the 4H3-7G antibodies respectively comprise the full length LC and HC of the amino acid sequences of SEQ ID NOs: 68 and 71 (1H3-7G) and SEQ ID NOs: 68 and 72 (4H3-7G), which are respectively encoded by the nucleotide sequences of SEQ ID NOs: 98 and 101 (1H3-7G) and SEQ ID NOs: 98 and 102 (4H3-7G).

According to yet another example of the present disclosure, the present antibody H3-8F is constructed into a hIgG1 vector, and is termed as the “1H3-8F” antibody. According to yet another example of the present disclosure, the present antibody H3-8F is constructed into a hIgG4 vector, and is termed as the “4H3-8F” antibody. The 1H3-8F and the 4H3-8F antibodies respectively comprise the full length LC and HC of the amino acid sequences of SEQ ID NOs: 68 and 73 (1H3-8F) and SEQ ID NOs: 68 and 74 (4H3-8F), which are respectively encoded by the nucleotide sequences of SEQ ID NOs: 98 and 103 (1H3-8F) and SEQ ID NOs: 98 and 104 (4H3-8F).

In a certain example of the present disclosure, the present antibody H3-9E is constructed into a hIgG1 vector, and is termed as the “1H3-9E” antibody. In another certain example of the present disclosure, the present antibody H3-9E is constructed into a hIgG4 vector, and is termed as the “4H3-9E” antibody. The 1H3-9E and the 4H3-9E antibodies respectively comprise the full length LC and HC of the amino acid sequences of SEQ ID NOs: 68 and 75 (1H3-9E) and SEQ ID NOs: 68 and 76 (4H3-9E), which are respectively encoded by the nucleotide sequences of SEQ ID NOs: 98 and 105 (1H3-9E) and SEQ ID NOs: 98 and 106 (4H3-9E).

In an optional example of the present disclosure, the present antibody H3-10E is constructed into a hIgG1 vector, and is termed as the “1H3-10E” antibody. In another optional example of the present disclosure, the present antibody H3-10E is constructed into a hIgG4 vector, and is termed as the “4H3-10E” antibody. The 1H3-10E and the 4H3-10E antibodies respectively comprise the full length LC and HC of the amino acid sequences of SEQ ID NOs: 68 and 77 (1H3-10E) and SEQ ID NOs: 68 and 78 (4H3-10E), which are respectively encoded by the nucleotide sequences of SEQ ID NOs: 98 and 107 (1H3-10E) and SEQ ID NOs: 98 and 108 (4H3-10E).

In a further example of the present disclosure, the present antibody H3-10F is constructed into a hIgG1 vector, and is termed as the “1H3-10F” antibody. In another further example of the present disclosure, the present antibody H3-10F is constructed into a hIgG4 vector, and is termed as the “4H3-10F” antibody. The 1H3-10F and the 4H3-10F antibodies respectively comprise the full length LC and HC of the amino acid sequences of SEQ ID NOs: 68 and 79 (1H3-10F) and SEQ ID NOs: 68 and 80 (4H3-10F), which are respectively encoded by the nucleotide sequences of SEQ ID NOs: 98 and 109 (1H3-10F) and SEQ ID NOs: 98 and 110 (4H3-10F).

According to some embodiments of the present disclosure, the present antibody S3H1-29 is constructed into a hIgG1 vector, and is termed as the “1S3H1-29” antibody. According to other embodiments of the present disclosure, the present antibody S3H1-29 is constructed into a hIgG4 vector, and is termed as the “4S3H1-29” antibody. The 1S3H1-29 and the 4S3H1-29 antibodies respectively comprise the full length LC and HC of the amino acid sequences of SEQ ID NOs: 68 and 81 (1S3H1-29) and SEQ ID NOs: 68 and 82 (4S3H1-29), which are respectively encoded by the nucleotide sequences of SEQ ID NOs: 98 and 111 (1S3H1-29) and SEQ ID NOs: 98 and 112 (4S3H1-29).

According to yet some embodiments of the present disclosure, the present antibody S3H1-74 is constructed into a hIgG1 vector, and is termed as the “1S3H1-74” antibody. According to yet other embodiments of the present disclosure, the present antibody S3H1-74 is constructed into a hIgG4 vector, and is termed as the “4S3H1-74” antibody. The 1S3H1-74 and the 4S3H1-74 antibodies respectively comprise the full length LC and HC of the amino acid sequences of SEQ ID NOs: 68 and 83 (1S3H1-74) and SEQ ID NOs: 68 and 84 (4S3H1-74), which are respectively encoded by the nucleotide sequences of SEQ ID NOs: 98 and 113 (1S3H1-74) and SEQ ID NOs: 98 and 114 (4S3H1-74).

According to still some embodiments of the present disclosure, the present antibody S3H1-76 is constructed into a hIgG1 vector, and is termed as the “1S3H1-76” antibody. According to still other embodiments of the present disclosure, the present antibody S3H1-76 is constructed into a hIgG4 vector, and is termed as the “4S3H1-76” antibody. The 1S3H1-76 and the 4S3H1-76 antibodies respectively comprise the full length LC and HC of the amino acid sequences of SEQ ID NOs: 68 and 85 (1S3H1-76) and SEQ ID NOs: 68 and 86 (4S3H1-76), which are respectively encoded by the nucleotide sequences of SEQ ID NOs: 98 and 115 (1S3H1-76) and SEQ ID NOs: 98 and 116 (4S3H1-76).

According to yet still some embodiments of the present disclosure, the present antibody S3H1-85 is constructed into a hIgG1 vector, and is termed as the “1S3H1-85” antibody. According to yet still other embodiments of the present disclosure, the present antibody S3H1-85 is constructed into a hIgG4 vector, and is termed as the “4S3H1-85” antibody. The 1S3H1-85 and the 4S3H1-85 antibodies respectively comprise the full length LC and HC of the amino acid sequences of SEQ ID NOs: 68 and 87 (1S3H1-85) and SEQ ID NOs: 68 and 88 (4S3H1-85), which are respectively encoded by the nucleotide sequences of SEQ ID NOs: 98 and 117 (1S3H1-85) and SEQ ID NOs: 98 and 119 (4S3H1-85).

Alternatively or in addition, the present antibody S3H2-8 is constructed into a hIgG1 vector, and is termed as the “1S3H2-8” antibody. Alternatively or in addition, the present antibody S3H2-8 is constructed into a hIgG4 vector, and is termed as the “4S3H2-8” antibody. The 1S3H2-8 and the 4S3H2-8 antibodies respectively comprise the full length LC and HC of the amino acid sequences of SEQ ID NOs: 68 and 89 (1S3H2-8) and SEQ ID NOs: 68 and 90 (4S3H2-8), which are respectively encoded by the nucleotide sequences of SEQ ID NOs: 98 and 119 (1S3H2-8) and SEQ ID NOs: 98 and 120 (4S3H2-8).

Optionally, the present antibody S3H2-10 is constructed into a hIgG1 vector, and is termed as the “1S3H2-10” antibody. Optionally, the present antibody S3H2-10 is constructed into a hIgG4 vector, and is termed as the “4S3H2-10” antibody. The 1S3H2-10 and the 4S3H2-10 antibodies respectively comprise the full length LC and HC of the amino acid sequences of SEQ ID NOs: 68 and 91 (1S3H2-10) and SEQ ID NOs: 68 and 92 (4S3H2-10), which are respectively encoded by the nucleotide sequences of SEQ ID NOs: 98 and 121 (1S3H2-10) and SEQ ID NOs: 98 and 122 (4S3H2-10).

Further, the present antibody S3H2-12 is constructed into a hIgG1 vector, and is termed as the “1S3H2-12” antibody. Further, the present antibody S3H2-12 is constructed into a hIgG4 vector, and is termed as the “4S3H2-12” antibody. The 1S3H2-12 and the 4S3H2-12 antibodies respectively comprise the full length LC and HC of the amino acid sequences of SEQ ID NOs: 68 and 93 (1S3H2-12) and SEQ ID NOs: 68 and 94 (4S3H2-12), which are respectively encoded by the nucleotide sequences of SEQ ID NOs: 98 and 123 (1S3H2-12) and SEQ ID NOs: 98 and 124 (4S3H2-12).

Moreover, the present antibody S3PL3-46 is constructed into a hIgG1 vector, and is termed as the “1S3PL3-46” antibody. Moreover, the present antibody S3PL3-46 is constructed into a hIgG4 vector, and is termed as the “4S3PL3-46” antibody. The 1S3PL3-46 and the 4S3PL3-46 antibodies respectively comprise the full length LC and HC of the amino acid sequences of SEQ ID NOs: 95 and 96 (1S3PL3-46) and SEQ ID NOs: 95 and 97 (4S3PL3-46), which are respectively encoded by the nucleotide sequences of SEQ ID NOs: 125 and 126 (1S3PL3-46) and SEQ ID NOs: 125 and 127 (4S3PL3-46).

Once produced, the humanized antibody may be purified according to standard procedures known in the art, such as cross-flow filtration, affinity column chromatography, gel filtration, etc. The humanized antibody shall perform in a manner identical or substantially similar to that of the non-human origin antibody.

The present recombinant antibody is specific to Siglec-3 receptor (i.e., the anti-Siglec-3 antibody or the anti-Siglec-3 antibody fragment), and comprises a VL region and a VH region, in which the VL region comprises a CDR-L1, a CDR-L2, and a CDR-L3, and the VH region comprises a CDR-H1, a CDR-H2, and a CDR-H3. The present anti-Siglec-3 recombinant antibody allows for slight sequence variations in each of the six CDRs, without compromising the overall binding specificity of the entire antibody (i.e., the combination of all the six CDRs) toward Siglec-3. As such, the present anti-Siglec-3 recombinant antibody may have
the CDR-L1 selected from the group consisting of SEQ ID NOs: 1 and 57;
the CDR-L2 selected from the group consisting of SEQ ID NOs: 2 and 58;
the CDR-L3 selected from the group consisting of SEQ ID NOs: 3 and 59;
the CDR-H1 selected from the group consisting of SEQ ID NOs: 5, 9, 13, 17, 21, 25, 29, 33, 37, 41, 45, 49, 53, and 61;
the CDR-H2 selected from the group consisting of SEQ ID NOs: 6, 10, 14, 18, 22, 26, 30, 34, 38, 42, 46, 50, 54, and 62; and
the CDR-H3 selected from the group consisting of SEQ ID NOs: 7, 11, 15, 19, 23, 27, 31, 35, 39, 43, 47, 51, 55, and 63.

According to some embodiments of the present disclosure, the present recombinant antibody is designated as 1H3-3A or 4H3-3A, in which the CDR-L1, the CDR-L2, and the CDR-L3 of the 1H3-3A or the 4H3-3A respectively have the amino acid sequences of SEQ ID NOs: 1, 2, and 3; and the CDR-H1, the CDR-H2, and the CDR-H3 of the 1H3-3A or the 4H3-3A respectively have the amino acid sequences of SEQ ID NOs: 5, 6, and 7. Preference is given to the VL region of the 1H3-3A or the 4H3-3A comprising an amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 4, and the VH region of the 1H3-3A or the 4H3-3A comprising an amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 8. As could be appreciated, the framework sequence of the VL and the VH regions may vary (e.g., being substituted by conserved or non-conserved amino acid residues) without affecting the binding affinity and/or specificity of the present antibody. Preferably, the sequences of the framework is conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue). According to preferred embodiments, the VL and the VH regions of the 1H3-3A or the 4H3-3A respectively comprise an amino acid sequence at least 90% identical to SEQ ID NOs: 4 and 8. More preferably, the VL and the VH regions of the 1H3-3A or the 4H3-3A respectively comprise an amino acid sequence at least 95% identical to SEQ ID NOs: 4 and 8. In one working example of the present disclosure, the VL and the VH regions of the 1H3-3A or the 4H3-3A respectively comprise the amino acid sequences of SEQ ID NOs: 4 and 8.

According to other embodiments of the present disclosure, the present recombinant antibody is designated as 1H3-7G or 4H3-7G, in which the CDR-L1, the CDR-L2, and the CDR-L3 of the 1H3-7G or the 4H3-7G respectively have the amino acid sequences of SEQ ID NOs: 1, 2, and 3; and the CDR-H1, the CDR-H2, and the CDR-H3 of the 1H3-7G or the 4H3-7G respectively have the amino acid sequences of SEQ ID NOs: 9, 10, and 11. Preferably, the VL and the VH region of the 1H3-7G or the 4H3-7G respectively comprise an amino acid sequence at least 85% identical to SEQ ID NOs: 4 and 12. More preferably, the VL and the VH regions of the 1H3-7G or the 4H3-7G respectively comprise an amino acid sequence at least 90% identical to SEQ ID NOs: 4 and 12. Even more preferably, the VL and the VH regions of the 1H3-7G or the 4H3-7G respectively comprise an amino acid sequence at least 95% identical to SEQ ID NOs: 4 and 12. In one working example of the present disclosure, the VL and the VH regions of the 1H3-7G or the 4H3-7G respectively comprise the amino acid sequences of SEQ ID NOs: 4 and 12.

According to certain embodiments of the present disclosure, the present recombinant antibody is designated as 1H3-8F or 4H3-8F, in which the CDR-L1, the CDR-L2, and the CDR-L3 of the 1H3-8F or the 4H3-8F respectively have the amino acid sequences of SEQ ID NOs: 1, 2, and 3; and the CDR-H1, the CDR-H2, and the CDR-H3 of the 1H3-8F or the 4H3-8F respectively have the amino acid sequences of SEQ ID NOs: 13, 14, and 15. Preferably, the VL and the VH region of the 1H3-8F or the 4H3-8F respectively comprise an amino acid sequence at least 85% identical to SEQ ID NOs: 4 and 16. More preferably, the VL and the VH regions of the 1H3-8F or the 4H3-8F respectively comprise an amino acid sequence at least 90% identical to SEQ ID NOs: 4 and 16. Even more preferably, the VL and the VH regions of the 1H3-8F or the 4H3-8F respectively comprise an amino acid sequence at least 95% identical to SEQ ID NOs: 4 and 16. In one working example of the present disclosure, the VL and the VH regions of the 1H3-8F or the 4H3-8F respectively comprise the amino acid sequences of SEQ ID NOs: 4 and 16.

In some embodiments, the present recombinant antibody is designated as 1H3-9E or 4H3-9E, in which the CDR-L1, the CDR-L2, and the CDR-L3 of the 1H3-9E or the 4H3-9E respectively have the amino acid sequences of SEQ ID NOs: 1, 2, and 3; and the CDR-H1, the CDR-H2, and the CDR-H3 of the 1H3-9E or the 4H3-9E respectively have the amino acid sequences of SEQ ID NOs: 17, 18, and 19. Preferably, the VL and the VH region of the 1H3-9E or the 4H3-9E respectively comprise an amino acid sequence at least 85% identical to SEQ ID NOs: 4 and 20. More preferably, the VL and the VH regions of the 1H3-9E or the 4H3-9E respectively comprise an amino acid sequence at least 90% identical to SEQ ID NOs: 4 and 20. Even more preferably, the VL and the VH regions of the 1H3-9E or the 4H3-9E respectively comprise an amino acid sequence at least 95% identical to SEQ ID NOs: 4 and 20. In certain working examples of the present disclosure, the VL and the VH regions of the 1H3-9E or the 4H3-9E respectively comprise the amino acid sequences of SEQ ID NOs: 4 and 20.

In certain embodiments, the present recombinant antibody is designated as 1H3-10E or 4H3-10E, in which the CDR-L1, the CDR-L2, and the CDR-L3 of the 1H3-10E or the 4H3-10E respectively have the amino acid sequences of SEQ ID NOs: 1, 2, and 3; and the CDR-H1, the CDR-H2, and the CDR-H3 of the 1H3-10E or the 4H3-10E respectively have the amino acid sequences of SEQ ID NOs: 21, 22, and 23. Preferably, the VL and the VH region of the 1H3-10E or the 4H3-10E respectively comprise an amino acid sequence at least 85% identical to SEQ ID NOs: 4 and 24. More preferably, the VL and the VH regions of the 1H3-10E or the 4H3-10E respectively comprise an amino acid sequence at least 90% identical to SEQ ID NOs: 4 and 24. Even more preferably, the VL and the VH regions of the 1H3-10E or the 4H3-10E respectively comprise an amino acid sequence at least 95% identical to SEQ ID NOs: 4 and 24. In some working examples of the present disclosure, the VL and the VH regions of the 1H3-10E or the 4H3-10E respectively comprise the amino acid sequences of SEQ ID NOs: 4 and 24.

In other embodiments, the present recombinant antibody is designated as 1H3-10F or 4H3-10F, in which the CDR-L1, the CDR-L2, and the CDR-L3 of the 1H3-10F or the 4H3-10F respectively have the amino acid sequences of SEQ ID NOs: 1, 2, and 3; and the CDR-H1, the CDR-H2, and the CDR-H3 of the 1H3-10F or the 4H3-10F respectively have the amino acid sequences of SEQ ID NOs: 25, 26, and 27. Preferably, the VL and the VH region of the 1H3-10F or the 4H3-10F respectively comprise an amino acid sequence at least 85% identical to SEQ ID NOs: 4 and 28. More preferably, the VL and the VH regions of the 1H3-10F or the 4H3-10F respectively comprise an amino acid sequence at least 90% identical to SEQ ID NOs: 4 and 28. Even more preferably, the VL and the VH regions of the 1H3-10F or the 4H3-10F respectively comprise an amino acid sequence at least 95% identical to SEQ ID NOs: 4 and 28. In some working examples of the present disclosure, the VL and the VH regions of the 1H3-10F or the 4H3-10F respectively comprise the amino acid sequences of SEQ ID NOs: 4 and 28.

In further embodiments, the present recombinant antibody is designated as 1S3H1-29 or 4S3H1-29, in which the CDR-L1, the CDR-L2, and the CDR-L3 of the 1S3H1-29 or the 4S3H1-29 respectively have the amino acid sequences of SEQ ID NOs: 1, 2, and 3; and the CDR-H1, the CDR-H2, and the CDR-H3 of the 1S3H1-29 or the 4S3H1-29 respectively have the amino acid sequences of SEQ ID NOs: 29, 30, and 31. Preferably, the VL and the VH region of the 1S3H1-29 or the 4S3H1-29 respectively comprise an amino acid sequence at least 85% identical to SEQ ID NOs: 4 and 32. More preferably, the VL and the VH regions of the 1S3H1-29 or the 4S3H1-29 respectively comprise an amino acid sequence at least 90% identical to SEQ ID NOs: 4 and 32. Even more preferably, the VL and the VH regions of the 1S3H1-29 or the 4S3H1-29 respectively comprise an amino acid sequence at least 95% identical to SEQ ID NOs: 4 and 32. In some working examples of the present disclosure, the VL and the VH regions of the 1S3H1-29 or the 4S3H1-29 respectively comprise the amino acid sequences of SEQ ID NOs: 4 and 32.

According to some embodiments, the present recombinant antibody is designated as 1S3H1-74 or 4S3H1-74, in which the CDR-L1, the CDR-L2, and the CDR-L3 of the 1S3H1-74 or the 4S3H1-74 respectively have the amino acid sequences of SEQ ID NOs: 1, 2, and 3; and the CDR-H1, the CDR-H2, and the CDR-H3 of the 1S3H1-74 or the 4S3H1-74 respectively have the amino acid sequences of SEQ ID NOs: 33, 34, and 35. Preferably, the VL and the VH region of the 1S3H1-74 or the 4S3H1-74 respectively comprise an amino acid sequence at least 85% identical to SEQ ID NOs: 4 and 36. More preferably, the VL and the VH regions of the 1S3H1-74 or the 4S3H1-74 respectively comprise an amino acid sequence at least 90% identical to SEQ ID NOs: 4 and 36. Even more preferably, the VL and the VH regions of the 1S3H1-74 or the 4S3H1-74 respectively comprise an amino acid sequence at least 95% identical to SEQ ID NOs: 4 and 36. In some working examples of the present disclosure, the VL and the VH regions of the 1S3H1-74 or the 4S3H1-74 respectively comprise the amino acid sequences of SEQ ID NOs: 4 and 36.

In still further embodiments, the present recombinant antibody is designated as 1S3H1-76 or 4S3H1-76, in which the CDR-L1, the CDR-L2, and the CDR-L3 of the 1S3H1-76 or the 4S3H1-76 respectively have the amino acid sequences of SEQ ID NOs: 1, 2, and 3; and the CDR-H1, the CDR-H2, and the CDR-H3 of the 1S3H1-76 or the 4S3H1-76 respectively have the amino acid sequences of SEQ ID NOs: 37, 38, and 39. Preferably, the VL and the VH region of the 1S3H1-76 or the 4S3H1-76 respectively comprise an amino acid sequence at least 85% identical to SEQ ID NOs: 4 and 40. More preferably, the VL and the VH regions of the 1S3H1-76 or the 4S3H1-76 respectively comprise an amino acid sequence at least 90% identical to SEQ ID NOs: 4 and 40. Even more preferably, the VL and the VH regions of the 1S3H1-76 or the 4S3H1-76 respectively comprise an amino acid sequence at least 95% identical to SEQ ID NOs: 4 and 40. In some working examples of the present disclosure, the VL and the VH regions of the 1S3H1-76 or the 4S3H1-76 respectively comprise the amino acid sequences of SEQ ID NOs: 4 and 40.

Moreover, the present recombinant antibody is designated as 1S3H1-85 or 4S3H1-85, in which the CDR-L1, the CDR-L2, and the CDR-L3 of the 1S3H1-85 or the 4S3H1-85 respectively have the amino acid sequences of SEQ ID NOs: 1, 2, and 3; and the CDR-H1, the CDR-H2, and the CDR-H3 of the 1S3H1-85 or the 4S3H1-85 respectively have an amino acid sequence of SEQ ID NOs: 41, 42, and 43. Preferably, the VL and the VH region of the 1S3H1-85 or the 4S3H1-85 respectively comprise an amino acid sequence at least 85% identical to SEQ ID NOs: 4 and 44. More preferably, the VL and the VH regions of the 1S3H1-85 or the 4S3H1-85 respectively comprise an amino acid sequence at least 90% identical to SEQ ID NOs: 4 and 44. Even more preferably, the VL and the VH regions of the 1S3H1-85 or the 4S3H1-85 respectively comprise an amino acid sequence at least 95% identical to SEQ ID NOs: 4 and 44. In some working example of the present disclosure, the VL and the VH regions of the 1S3H1-85 or the 4S3H1-85 respectively comprise the amino acid sequences of SEQ ID NOs: 4 and 44.

In some embodiments, the present recombinant antibody is designated as 1S3H2-8 or 4S3H2-8, in which the CDR-L1, the CDR-L2, and the CDR-L3 of the 1S3H2-8 or the 4S3H2-8 respectively have the amino acid sequences of SEQ ID NOs: 1, 2, and 3; and the CDR-H1, the CDR-H2, and the CDR-H3 of the 1S3H2-8 or the 4S3H2-8 respectively have the amino acid sequences of SEQ ID NOs: 45, 46, and 47. Preferably, the VL and the VH region of the 1S3H2-8 or the 4S3H2-8 respectively comprise an amino acid sequence at least 85% identical to SEQ ID NOs: 4 and 48. More preferably, the VL and the VH regions of the 1S3H2-8 or the 4S3H2-8 respectively comprise an amino acid sequence at least 90% identical to SEQ ID NOs: 4 and 48. Even more preferably, the VL and the VH regions of the 1S3H2-8 or the 4S3H2-8 respectively comprise an amino acid sequence at least 95% identical to SEQ ID NOs: 4 and 48. In some working examples of the present disclosure, the VL and the VH regions of the 1S3H2-8 or the 4S3H2-8 respectively comprise the amino acid sequences of SEQ ID NOs: 4 and 48.

In yet some embodiments, the present recombinant antibody is designated as 1S3H2-10 or 4S3H2-10, in which the CDR-L1, the CDR-L2, and the CDR-L3 of the 1S3H2-10 or the 4S3H2-10 respectively have the amino acid sequences of SEQ ID NOs: 1, 2, and 3; and the CDR-H1, the CDR-H2, and the CDR-H3 of the 1S3H2-10 or the 4S3H2-10 respectively have the amino acid sequences of SEQ ID NOs: 49, 50, and 51. Preferably, the VL and the VH region of the 1S3H2-10 or the 4S3H2-10 respectively comprise an amino acid sequence at least 85% identical to SEQ ID NOs: 4 and 52. More preferably, the VL and the VH regions of the 1S3H2-10 or the 4S3H2-10 respectively comprise an amino acid sequence at least 90% identical to SEQ ID NOs: 4 and 52. Even more preferably, the VL and the VH regions of the 1S3H2-10 or the 4S3H2-10 respectively comprise an amino acid sequence at least 95% identical to SEQ ID NOs: 4 and 52. In some working examples of the present disclosure, the VL and the VH regions of the 1S3H2-10 or the 4S3H2-10 respectively comprise the amino acid sequences of SEQ ID NOs: 4 and 52.

According to other embodiments, the present recombinant antibody is designated as 1S3H2-12 or 4S3H2-12, in which the CDR-L1, the CDR-L2, and the CDR-L3 of the 1S3H2-12 or the 4S3H2-12 respectively have the amino acid sequences of SEQ ID NOs: 1, 2, and 3; and the CDR-H1, the CDR-H2, and the CDR-H3 of the 1S3H2-12 or the 4S3H2-12 respectively have the amino acid sequences of SEQ ID NOs: 53, 54, and 55. Preferably, the VL and the VH region of the 1S3H2-12 or the 4S3H2-12 respectively comprise an amino acid sequence at least 85% identical to SEQ ID NOs: 4 and 56. More preferably, the VL and the VH regions of the 1S3H2-12 or the 4S3H2-12 respectively comprise an amino acid sequence at least 90% identical to SEQ ID NOs: 4 and 56. Even more preferably, the VL and the VH regions of the 1S3H2-12 or the 4S3H2-12 respectively comprise an amino acid sequence at least 95% identical to SEQ ID NOs: 4 and 56. In some working examples of the present disclosure, the VL and the VH regions of the 1S3H2-12 or the 4S3H2-12 respectively comprise the amino acid sequences of SEQ ID NOs: 4 and 56.

According to other embodiments, the present recombinant antibody is designated as 1S3PL3-46 or 4S3PL3-46, in which the CDR-L1, the CDR-L2, and the CDR-L3 of the 1S3PL3-46 or the 4S3PL3-46 respectively have the amino acid sequences of SEQ ID NOs: 57, 58, and 59; and the CDR-H1, the CDR-H2, and the CDR-H3 of the 1S3PL3-46 or the 4S3PL3-46 respectively have the amino acid sequences of SEQ ID NOs: 61, 62, and 63. Preferably, the VL and the VH region of the 1S3PL3-46 or the 4S3PL3-46 respectively comprise an amino acid sequence at least 85% identical to SEQ ID NOs: 60 and 64. More preferably, the VL and the VH regions of the 1S3PL3-46 or the 4S3PL3-46 respectively comprise an amino acid sequence at least 90% identical to SEQ ID NOs: 60 and 64. Even more preferably, the VL and the VH regions of the 1S3PL3-46 or the 4S3PL3-46 respectively comprise an amino acid sequence at least 95% identical to SEQ ID NOs: 60 and 64. In some working examples of the present disclosure, the VL and the VH regions of the 1S3PL3-46 or the 4S3PL3-46 respectively comprise the amino acid sequences of SEQ ID NOs: 60 and 64.

### 2. The Pharmaceutical Composition

The present disclosure also encompasses a pharmaceutical composition comprising the above recombinant antibody for use in preventing or treating HBV infection, a neurodegenerative disease, an autoimmune disease, or a cancer, and/or alleviating or ameliorating the symptoms associated with/caused by HBV infection, a neurodegenerative disease, an autoimmune disease, or a cancer. Specifically, the pharmaceutical composition comprises an effective amount of the recombinant antibody as described in any aspects or embodiments of the present disclosure; and optionally, a pharmaceutically acceptable excipient. The ratio of the recombinant antibody in the pharmaceutical composition by weight is as described above, and the detailed description is omitted herein for the sake of brevity.

The present pharmaceutical composition is prepared following the pharmaceutical procedures known in the art, and may be formulated into solid, semi-solid, or liquid forms, such as tablets, capsules, powders, granules, ointments, solutions, suspensions, and injections, compatible with the intended routes of administration. These pharmaceutical preparations are a further object of the invention. One of skilled person in the art is familiar with the various dosage forms that are suitable for use in each route. It is to be noted that the most suitable route in any given case would depend on the nature or severity of the disease or condition being treated.

A pharmaceutically acceptable excipient is any excipient which is relatively non-toxic and innocuous to a patient at concentrations consistent with effective activity of the active ingredient so that any side effects ascribable to the excipient do not vitiate the beneficial effects of the active ingredient. Pharmaceutically acceptable excipients according to the invention are for example disintegrants, binders, lubricants, fillers, plasticizers, surfactants and wetting agents, film-forming agents and coating materials, and coloring agents for example pigments.

Disintegrants include, but are not limited to, croscarmellose sodium, crospovidone, alginic acid, carboxymethylcellulose calcium, carboxymethylcellulose sodium, microcrystalline cellulose, hydroxypropyl cellulose, low substituted hydroxypropyl cellulose, polacrillin potassium, cross-linked polyvinylpyrrolidone, sodium alginate, sodium starch glycollate, partially hydrolysed starch, sodium carboxymethyl starch and starch. Preference is given to croscarmellose sodium and/or cross-linked polyvinylpyrrolidone, more preference is given to croscarmellose sodium.

Binders include, but are not limited to, hydroxypropyl cellulose, hypromellose (hydroxypropyl methylcellulose, HPMC), microcrystalline cellulose, acacia, alginic acid, carboxymethylcellulose, ethylcellulose, methylcellulose, hydroxaethylcellulose, ethylhydroxyethylcellulose, polyvinyl alcohol, polyacrylates, carboxymethylcellulose calcium, carboxymethylcellulose sodium, compressible sugar, ethylcellulose, gelatin, liquid glucose, methylcellulose, polyvinyl pyrrolidone and pregelatinized starch.

Lubricants include, but are not limited to, calcium stearate, magnesium stearate, mineral oil, stearic acid, fumaric acid, sodium stearylfumarate, zinc stearate and polyethyleneglycol.

Fillers include, but are not limited to, dibasic calcium phosphate, kaolin, lactose, mannitol, micro-crystalline cellulose, silicated microcrystalline cellulose, dicalcium phosphate, tricalcium phosphate, magnesium trisilicate, mannitol, maltitol, sorbitol, xylitol, lactose for example the anhydrous form or the hydrate form such as the monohydrate form, dextrose, maltose, saccharose, glucose, fructose or maltodextrine, powdered cellulose, precipitated calcium carbonate, sodium carbonate, sodium phosphate and starch.

Surfactants and wetting agents include, but are not limited to, heptadecaethylene oxycetanol, lecithins, sorbitol monooleate, polyoxyethylene sorbitol monooleate, polyoxyethylene stearate, polyoxyethylen sorbitan monolaurate, benzalkonium chloride, nonoxynol 10, oxtoxynol 9, polysorbates for example 20, 40, 60 or 80, sorbitan mono-palmitate, sodium salts of fatty alcohol-sulfates such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (PLURONIC©) and ethoxylated triglycerides.

Film-forming agents and coating materials include, but are not limited to, liquid glucose, hydroxy-ethyl cellulose, hydroxypropyl cellulose, hydroxypropyl methylcellulose (hypromellose, HPMC), methylcellulose, ethylcellulose, cellulose acetate phthalate, shellac, polyvinylpyrrolidone, copolymers of vinylpyrrolidone and vinylacetate such as Kollidon© VA64 BASF, copolymers of acrylic- and/or methacrylic acid esters with trimethylammoniummethylacrylate, copolymers of dimethylaminomethacrylic acid and neutral methacrylic acid esters, polymers of methacrylic acid or methacrylic acid esters, copolymers of acrylic acid ethylester and methacrylic acid methyl ester, and copolymers of acrylic acid and acrylic acid methylester.

Plasticizers include, but are not limited to polyethylene glycol, diethyl phthalate and glycerol.

Coloring agents include, but are not limited to pigments, inorganic pigments, FD&C Red No. 3, FD&C Red No. 20, FD&C Yellow No. 6, FD&C Blue No. 2, D&C Green No. 5, D&C Orange No. 5, D&C Red No. 8, caramel, ferric oxide red, ferric oxide yellow and titanium dioxide. Preference is given to ferric oxide red, ferric oxide yellow and titanium dioxide.

Further commonly used pharmaceutical excipients which can be used as appropriate to formulate the composition for its intended route of administration include, but is not limited to, acidifying agents (e.g., acetic acid, citric acid, fumaric acid, hydrochloric acid, and nitric acid); alkalizing agents (e.g., ammonia solution, ammonium carbonate, diethanolamine, mono-ethanolamine, potassium hydroxide, sodium borate, sodium carbonate, sodium hydroxide, triethanolamine, and trolamine); adsorbents (e.g., powdered cellulose, and activated charcoal); stabilizers and antioxidants (e.g., ascorbic acid, ascorbyl palmitate, butylated hydroxy-anisole, butylated hydroxytoluene, hypophosphorus acid, monothioglycerol, propyl gallate, sodium ascorbate, sodium bisulfite, sodium formaldehyde sulfoxylate, and sodium metabisulfite); other binding materials (e.g., block polymers, natural and synthetic rubber, polyacrylates, polyurethanes, silicones, polysiloxanes, and styrene-butadiene copolymers); buffering agents (e.g., potassium metaphosphate, dipotassium phosphate, sodium acetate, sodium citrate anhydrous, and sodium citrate hydrates); encapsulating agents (e.g., gelatin, starch, and cellulose derivates); flavorants, masking agents and odors (e.g., anise oil, cinnamon oil, cocoa, menthol, orange oil, peppermint oil, and vanillin); humectants (e.g., glycerol, propylene glycol, and sorbitol); sweeteners (e.g., aspartame, dextrose, glycerol, mannitol, propylene glycol, saccharin sodium, sorbitol, and sucrose); anti-adherents (e.g., magnesium stearate, and talc); direct compression excipients (e.g., dibasic calcium phosphate, lactose, and microcrystalline cellulose); tablet polishing agents (e.g., carnauba wax, and white wax).

The present pharmaceutical composition may further comprise other known pharmaceutically active agents (e.g., an antiviral agent) to treat diseases and conditions caused by HBV infection. Such antiviral agent may be acyclovir, adefovir, brincidofovir, brivudine, cidofovir, clevudine, cytarabine, docosanol, edoxudine, entecavir, famciclovir, filociclovir, fomivirsen, foscarnet, ganciclovir, idoxuridine, imiquimod, interferon-α, lamivudine, lobucavir, maribavir, methisazone, moroxydine, penciclovir, peginterferon-α, podophyllotoxin, ribavirin, rifampicin, resiquimod, sorivudine, taribavirin, tecovirimat, telbivudine, tenofovir, thymosin-α, trifluridine, tromantadine, valaciclovir, valganciclovir, vesatolimod (GS-9620), vidarabine, viread, and a combination thereof. Preference is given to vesatolimod (GS-9620).

Alternatively, the present pharmaceutical composition may further comprise other pharmaceutical agents (e.g., a neurodegenerative disease treatment agent) for treating diseases and conditions associated with a neurodegenerative disease. Examples of such neurodegenerative disease treatment agents include, but are not limited to, acyclovir, aducanumab, amantadine, apomorphine, baclofen, carbidopa, carbidopa, dantrolene, donepezil, entacapone, foscarnet, galantamine, levodopa, memantine, penciclovir, pramipexole, rasagiline, riluzole, rivastigmine, ropinirole, selegiline, tacrine, tetrabenazine, tizanidine, or tolcapone.

Optionally, the present pharmaceutical composition may further comprise other pharmaceutical agents (e.g., an agent for treating autoimmune disease) for treating diseases and conditions associated with an autoimmune disease. Suitable autoimmune disease treatment agents may be abatacept, acitretin, adalimumab, alefacept, anakinra, anthralin, apremilast, azathioprine, baricitinib, belimumab, benralizumab, betamethasone, bimekizumab, brazikumab, brodalumab, calcipotriene, calcipotriol, calcitriol, canakinumab, certolizumab, clobetasol, coal tar, colchicine, cyclosporine, dapsone, dithranol, dexamethasone, dupilumab, eculizumab, etanercept, fluocinolone, golimumab, guselkumab, hydroxychloroquine, hydrocortisone, infliximab, ixekizumab, lenercept, mepolizumab, methotrexate, methylprednisolone, mirikizumab, mycophenolate mofetil, omalizumab, prednisone, perakizumab, pimecrolimus, remtolumab, reslizumab, rilonacept, risankizumab, rituximab, sarilumab, secukinumab, sulfasalazine, tacrolimus, tazarotene, tildrakizumab, tocilizumab, tofacitinib, tretinoin, upadacitinib, ustekinumab, vedolizumab, or vunakizumab.

Still optionally, the present pharmaceutical composition may further comprise other pharmaceutical agents (e.g., an anti-cancer drug) for treating diseases and conditions associated with a cancer. Said anti-cancer drugs are selected at least one from the group consisting of actinomycin D, altretamine, aminoglutethimide, amsacrin, anastrozole, anthracycline, asparaginase, bendamustine, bexarotene, bleomycin, bortezomib, buserelin, busulfan, camptothecin, capecitabine, carboplatin, carmustine, chlorambucil, chlormethine, cisplatin, cladribine, clofarabine, clomifene, curcumin, cyclophosphamide, cytarabine, cytosinarabinoside, dacarbazine, dactinomycin, daunorubicin, dexamethasone, docetaxel, doxorubicin, epirubicin, estramustine, estrone, estradiol, estriol, etoposide, etoposid, exemestane, fludarabine, fluorouracil, formestane, foxuridine, gemcitabine, glucocorticoid, goserelin, hycamtin, hydroxy urea, idarubicin, ifosfamid, imatinib, indirubin, irinotecan, ixabepilone, letrozole, leuprorelin, lomustine, mechlorethamine, melphalan, mercaptopurine, methotrexate, miltefosin, mitomycine, mitoxantrone, nintedanib, nimustine, oxaliplatin, paclitaxel, pentostatin, plicamycin, prednisone, procarbazine, progesterone, raloxifene, streptozocin, tamoxifen, temozolomide, teniposide, testolactone, testosterone, thiotepa, thioguanine, topotecan, treosulfan, tretinoin, triptorelin, trofosfamide, vinblastine, vincristine, vindesine, and vinorelbine.

3. The Method for Preventing and/or Treating Siglec-3 Associated Diseases

Another aspect of the present disclosure pertains to a method of preventing and/or treating HBV infection, a neurodegenerative disease, an autoimmune disease, or a cancer in a subject. The method comprises administering to the subject an effective amount of the present recombinant antibody, or the pharmaceutical composition of the present disclosure.

According to the embodiments of the present disclosure, activation of Siglec-3 may lead to suppression of the innate immune response of the subject through the Siglec-3 receptor-mediated signaling pathway, and administering the present recombinant antibody (i.e., the anti-Siglec-3 antibody) may reverse the immunosuppression by blocking the binding between Siglect-3 ligand (e.g., sialic acid-containing ligand) and Siglect-3 on the immune (e.g., dendritic cells), thereby activating/improving the host immunity against Siglect-3 associated diseases. Accordingly, the present recombinant anti-Siglec-3 antibody may serve as a potential means to prevent or treat the diseases associated with Siglect-3 over activation, and/or alleviate or ameliorate the symptoms associated with/caused by such diseases.

The effective dose administered to the subject is from about 0.01 to 1,000 mg/kg body weight of the subject, such as 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 mg/kg body weight of the subject; preferably, about 0.1 to 100 mg/kg body weight of the subject. The dose can be administered in a single aliquot, or alternatively in more than one aliquot. The skilled artisan or clinical practitioner may adjust the dosage or regime in accordance with the physical condition of the patient or the severity of the diseases.

As would be appreciated, in the case when the subject afflicted with HBV infection, the present method may be applied to the subject alone or in combination with additional therapies (e.g., an antiviral agent) that have some beneficial effects on the prevention or treatment of HBV infection. Depending on the intended therapeutic purposes, the present method may be applied to the subject prior to, in conjunction with, or subsequent to the administration of the additional therapies. Said additional therapies (e.g., an antiviral agent) are as described above; for the sake of brevity, the examples of the antiviral agent are omitted herein.

Alternatively, the present method may be used to treat the subject suffered from a neurodegenerative disease. Said neurodegenerative disease may be any one of Alzheimer's disease, Batten disease, Parkinson's disease, dementia with Lewy bodies, multiple system atrophy, bovine spongiform encephalopathy, Creutzfeldt-Jakob disease, Huntington's disease, amyotrophic lateral sclerosis, ataxia telangiectasia, multiple sclerosis, spinocerebellar atrophy, HIV-associated neurocognitive disorders, Pick's disease, Krabbe's disease, spinal and bulbar muscular atrophy, primary lateral sclerosis, Cockayne syndrome, spinal muscular atrophy, tabes dorsalis, progressive supranuclear palsy, or Pelizaeus-Merzbacher disease. In one working example, the neurodegenerative disease treatable with the present method is Alzheimer's disease. Accordingly, in the case when the present method is used to treat the neurodegenerative disease, the present method may further comprise administering to the subject an effective amount of another agent for treating neurodegenerative disease as described above.

According to some optional embodiments of the present disclosure, the present method may be used in treating an autoimmune disease in a subject; such autoimmune disease may be acute disseminated encephalomyelitis, Addison's disease, alopecia areata, antiphospholipid syndrome, antisynthetase syndrome, asthma, autoimmune angioedema, autoimmune hepatitis, autoimmune pancreatitis, autoimmune polyendocrine syndrome type 1-3, autoimmune progesterone dermatitis, autoimmune thyroiditis, autoimmune urticaria, bullous pemphigoid, chronic hives, cicatricial pemphigoid, coeliac disease, Crohn's disease, dermatitis herpetiformis, dermatomyositis, diabetes mellitus type 1, discoid lupus erythematosus, endometriosis, epidermolysis bullosa acquisita, esophageal achalasia, erythema nodosum, familial mediterranean fever, gestational pemphigoid, Graves' disease, gout, hidradenitis suppurativa, IgAvasculitis, inflammatory bowel disease, interstitial cystitis, lichen planus, lichen sclerosus, lupus nephritis, morphea, Mucha-Habermann disease, Muckle-Wells syndrome, myocarditis, myasthenia gravis, pemphigus vulgaris, pityriasis lichenoides et varioliformis acuta, postmyocardial infarction syndrome, primary biliary cholangitis, primary sclerosing cholangitis, psoriasis, rheumatoid arthritis, Sjögren syndrome, subacute bacterial endocarditis, systemic lupus erythematosus (SLE), systemic scleroderma, ulcerative colitis, or vitiligo. In a specific example, the autoimmune disease treatable with the present method is SLE. In accordance with the treatment for the autoimmune disease, the present method may additionally comprise administering to the subject another autoimmune disease treatment agent as described above.

Optionally, the present method may be used in treating a subject suffered from a cancer, such as bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer. Thus, the present method may include applying another agent, an anti-cancer drug, to the subject, in which the examples of anti-cancer drug are those as described above, and are omitted herein for the sake of brevity.

It should be noted that during the term of the present treatment, different therapies or therapeutics may be administered to the subject at different doses, time intervals, via different routes. The doses and time intervals may vary with factors as described above, and are dependent on the professional considerations of the practitioner; and the routes may be via oral, enteral, buccal, nasal, transdermal, transmucosal, intravenous, intraperitoneal, intraarterial, intracutaneous, subcutaneous, and intramuscular routes.

Basically, the subject treatable by the present method is a mammal; and preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

1. Screening of Anti-Siglec-3 Monoclonal scFvs from the Phage-Displayed scFv Libraries

### 1.1 Phage Display Selection-Amplification Cycles

The recombinant extracellular domain of human Siglec-3 was fused with Fc portion of hIgG1 (Siglec-3.Fc), which was then served as a target for phage screening. The Siglec-3.Fc was coated on a 96-well microplate (5 μg per well), and then the microplate was subjected to blocked with 5% skim milk in PBST (1×PBS with 0.05% (v/v) Tween 20, pH 7.4) for 1 hour. After blocking, 100 μL of the resuspended polyethylene glycol/NaCl-precipitated phage display library (˜10¹³ CFU/mL in 5% skim milk in PBST) was added to each well for 1 hour. The microplate was then washed 6 times with 300 μL PBST and 3 times with 300 μL PBS. The bound phages in each well of the microplate were eluted with 100 μL of 0.1 M HCl/glycine (pH 2.2) per well, and immediately neutralized with 8 μL of 2 M Tris-base buffer (pH 9.1). The eluted phages were amplified in E. coli ER2738 (A600 nm=0.6) for 30 minutes at 37° C. in the presence of 100 g/mL ampicillin. The bacterial culture was further incubated with 100 L of M13KO7 helper phage (^{˜}10¹¹ CFU total) at 37° C. for 1 hour, and then cultured in a 50 mL of 2×YT medium containing 50 g/mL kanamycin and 100 g/mL ampicillin overnight at 37° C. The resultant rescued phages were precipitated with polyethylene glycol/NaCl, and then resuspended in PBS. The concentrated phage solution containing the rescued phages was used for the next round of biopanning.

1.2 Polyclonal Soluble scFvs in E. coli Culture Media Evaluated for Antigen Binding with ELISA

50 μL of the concentrated phage solution containing the rescued phages from each cycle of biopanning as described above was mixed with 750 μL of E. coli ER2738 (A600 nm=0.6) in a 96-well microplate, and the culture was incubated at 37° C. for 1 hour. Then, 100 μL of ampicillin was added to reach the final concentration of 100 μg/mL. When A600 nm>1.0, 100 μL of 10 mM IPTG was added to each well to reach the final concentration of 1 mM, and the microplate was then incubated at 37° C. overnight. The microplate was centrifuged at 3,000×g for 10 minutes, and the supernatants containing the secreted polyclonal soluble scFvs were used for ELISA binding assay below.

1.3 ELISA Assay for Soluble scFv-Antigen Binding

After 2-3 rounds of biopanning cycles, several single phage colonies were selected, and each soluble monoclonal scFv from each phage colony was prepared by similar procedures as described above (i.e., induction by IPTG in the E. coli cultures); these monoclonal scFvs were respectively designated as H3-3A, H3-7G, H3-8F, H3-9E, H3-10E, H3-10F, S3H1-29, S3H1-74, S3H1-76, S3H1-85, S3H2-8, S3H2-10, S3H2-12, and S3PL3-46. The binding of the soluble monoclonal scFv to its antigen was analyzed by ELISA assay according to the following procedures: coating a microplate with Siglec-3.Fc (0.5 μg/well), and blocking unspecific binding by use of 5% skim milk in PBST for 1 hour; and adding 100 μL of the supernatants containing the secreted scFvs to the microplate. After incubation for 1 hr, 100 μL of anti E-tag-HRP (1:4000 dilution) was added to each well and incubated for another hour. The microplate was washed, developed with 3,3′,5,5′-tetramethyl-benzidine peroxidase substrate (TMB substrate), and quenched with 1.0 M HCl. The microplate was then read at OD450 nm.

### 1.4 Selection of Cell Surface-Bound Antibody by Flow Cytometry

For selection of cell surface-bound antibodies, 293T cells expressing the full length of the Siglec-3-EGFP on the cell surface were prepared for the purpose. Briefly, 293T cells were transfected with the full length Siglec-3-EGFP construct by lipofectamine 2000 (Thermo Fisher Scientific), and incubated for 48 hours. After that, the cells were incubated with the monoclonal scFvs for 30 minutes, followed by incubation with the scFv binding protein-RFP fusion protein for another 30 minutes. The cells were then fixed by 2% of paraformaldehyde, and analyzed by flow cytometry (FACSVerse; BD Biosciences).

2. Reformation of scFvs into IgG1s or IgG4s

The monoclonal scFvs (i.e., H3-3A, H3-7G, H3-8F, H3-9E, H3-10E, H3-10F, S3H1-29, S3H1-74, S3H1-76, S3H1-85, S3H2-8, S3H2-10, S3H2-12, and S3PL3-4) were reformatted into IgG1s or IgG4s via conventional molecular cloning technologies, in which the VL region of each scFv was fused with the CK moiety (SEQ ID NO: 65), and the VH region of each scFv was fused with the IgG1 CH moiety (SEQ ID NO: 66) to give the monoclonal IgG1s that were respectively termed as 1H3-3A, 1H3-7G, 1H3-8F, 1H3-9E, 1H3-10E, 1H3-10F, 1S3H1-29, 1S3H1-74, 1S3H1-76, 1S3H1-85, 1S3H2-8, 1S3H2-10, 1S3H2-12, and 1S3PL3-46. Alternatively, the VH region of each scFv was fused with the IgG4 CH moiety (SEQ ID NO: 67) to give the monoclonal IgG4s that were respectively termed as 4H3-3A, 4H3-7G, 4H3-8F, 4H3-9E, 4H3-10E, 4H3-10F, 4S3H1-29, 4S3H1-74, 4S3H1-76, 4S3H1-85, 4S3H2-8, 4S3H2-10, 4S3H2-12, and 4S3PL3-46.

### 3. Size-Exclusion Chromatography

For purification, the above anti-Siglec-3 antibodies, as well as two other anti-Siglec-3 antibodies 10C8 and 2B9 (as positive controls) were applied to a gel filtration column (GE Healthcare), and the purification was performed on an equipment of size-exclusion chromatography (AKTA™ systems).

### 4. Cell Cultures

### 4.1 Preparation of Human Primary Cells

For preparation of PBMCs, the whole blood from healthy human donors or CHB patients was isolated, and subjected to the standard density-gradient centrifugation using a density gradient media (Ficoll-Paque; Amersham Biosciences). For preparation of monocytes, a cell separator system using magnetic sorting (VarioMACS) was deploied with anti-CD14 microbeads (Miltenyi Biotec GmbH) to isolate CD14⁺ cells. After isolation, cells were then incubated in complete RPMI 1640 medium supplemented with 10% fetal calf serum and human GM-CSF/IL-4 at 37° C. under a humidified atmosphere of 5% CO₂ for 6-7 days to generate moDCs.

### 4.2 Preparation of Siglec-3-Expressing HEK-293T Cells

HEK-293T cells were transfected with pcDNA3-human CD33 (Siglec-3) plasmids for 48 hours before harvest for FACS staining.

4.3 Preparation of iPS-Derived Human Microglia

The iPS were incubated with mTeSR medium supplemented with BMP4, VEGF, and SCF for four days, and then transferred to plates and incubated with X-VIVO 15 supplemented with IL-3, MCF, Glutamax, and 3-mercaptoethanol for after Day 7 till being harvested.

### 4.4 Preparation of SLE Patient-Derived EVs

The blood samples of SLE patients were subjected to ulctracentrifugation to purify EVs from sera.

### 5. Flow Cytometry

For establishment of titration curves, human monocytes and moDCs (3×10⁴) were incubated with the indicated concentrations of the APC-conjugated anti-Siglec-3 antibodies 4H3-3A, 10C8, as well as hIgG1 control, and analyzed by a flow cytometry instrument (FACSVerse; BD Biosciences). Mean fluorescence intensity (MFI) was analyzed by a software FlowJo (TreeStar, Inc.).

For analyzing the surface marker expressions, PBMCs (1×10⁶) from healthy human donors and CHB patients were treated with the anti-Siglec-3 antibodies 4H3-3A and 10C8, as well as hIgG1 control (3 μg/mL), followed by GS-9620 treatment (10 nM) for another 24 hours or not. The cells were harvested and incubated with anti-Siglec-3, anti-CD80, anti-CD86, anti-PD-L1, anti-MHC-I, and anti-MHC-II antibodies. The binding measured by flow cytometry (FACSVerse; BD Biosciences) and analyzed by FlowJo (TreeStar, Inc.).

### 6. Biacore Analysis

Human IgG1 was first mobilized on channel one of a CM5 chip as the blank to determine the bulk effect of injection itself, whereas human CD33.Fc were immobilized on channel two for analysis of its kinetic interaction with wild various clones of anti-CD33 mAbs, respectively. The interaction between DcR3.Fc and anti-CD33 mAbs was determined by surface plasmon resonance using a Biacore instrument (BIAcore 2000).

### 7. ELISA

For analyzing the cytokine secretions, PBMCs (1×10⁶) from healthy human donors and CHB patients were treated with the anti-Siglec-3 antibodies 4H3-3A and 10C8, as well as hIgG1 control (3 μg/mL), followed by stimulation with GS-9620 treatment (10 nM and 30 nM) for another 24 hours or not. The supernatants were collected, and the cytokine production level of IFN-α, TNF-α, and IL-6 was measured by ELISA kits (R&D, and Thermo Fisher Scientific).

The antibody against HBsAg (HBsAb) in PBMCs-cultured medium was detected by HBsAg ELISA kit (Creative Diagnostics).

### 8. ELISpot

For HBsAb ELISPOT, recombinant HBsAg was coated on methanol-rinsed MultiScreen-IP Filter Plate, followed by incubation with PBMCs (2.5×10⁵) for 24 hours, then adding to Biotin-conjugated goat anti-human IgG antibody and HRP Streptavidin sequentially. Finally, AEC Substrate was added to detect the numbers of spots on the plates.

### 9. Phargocytosis Evaluation

For evaluation of the Aβ₁₋₄₂ or Tau phargocytosis by iPS-derived microglia, the iPS-derived microglia were pre-incubated with the isotype control antibody (hIgG1) or anti-human Siglec-3 antibodies 2B9, 10C8, and 4H3-3A at the indicated concentrations, prior to incubated with Aβ₁₋₄₂ oligomer (5 or 20 g/ml) or Tau protein (20 g/ml) at 37° C. for 1 hour. Then, the cells were fixed and stained with Hochest33342 and phalloidin to visualize the DNA and the cell membrane, respectively. The level of the Aβ₁₋₄₂ or Tau phagocytosis was measured using flow cytometry (BDverse) and shown as percentage (%) of phargocytosis and mean fluorescence (MFI).

### 10. NET Formation Assessment

To measure NET formation, samples were fixed with 4% paraformaldehyde and then permeabilized with 0.5% Triton X100 in PBS for 15 min. Components of NETs were visualized by staining with anti-MPO antibody (2 μg/ml), anti-citrullinated histone antibody (3 μg/ml), and Hoechst 33342 (0.5 μg/ml). The histone area of NETs was determined from images captured using a confocal microscope with white light laser system (TCS SP8 X-FALCON, Leica) and analyzed using the MetaMorph™ software.

### 11. Statistics

Results were expressed as mean±standard deviation (S.D.). Two-way ANOVA was used for statistical comparisons between experiment and control groups. Differences are considered significant at p<0.05, vs. control.

### Example 1 Preparation of the Monoclonal Anti-Siglec-3 Antibodies

The aim of the example is to pursue novel anti-human Siglec-3 monoclonal antibodies that may possess a potential ability to inhibit the HBV-mediated immunosuppression. To achieve the foregoing aim, the means by using the phage-displayed scFv libraries were adopted following the procedures as described in the section of “Materials and Methods.” After completion of the biopanning rounds, several candidates of the anti-Siglec-3 monoclonal scFv clones were selected (i.e., H3-3A, H3-7G, H3-8F, H3-9E, H3-10E, H3-10F, S3H1-29, S3H1-74, S3H1-76, S3H1-85, S3H2-8, S3H2-10, S3H2-12, and S3PL3-4), reformatted into human IgG1 or IgG4, and subjected to evaluation of their inhibitory effects on HBV-mediated immunosuppression. Among the selected candidates, clones 2B9, 10C8, and 4H3-3A (i.e., the scFv clone H3-3A reformatted into hIgG4) displayed the highest mean fluorescence intensity (MFI) for binding to monocyte-derived dendritic cells (moDCs) by flow cytometry (data not shown), while the other 13 anti-Siglec-3 antibodies also exhibited similar binding activities toward moDCs (data not shown).

### Example 2 Characterization of the Anti-Siglec-3 Antibody 4H3-3A of Example 1

In this example, all the anti-Siglec-3 antibodies of Example 1 had been characterized for their immunomodulating ability on host immune system (i.e., protecting the host from HBV infection). Note that for the sake of brevity, only data for the anti-Siglec-3 antibody 4H3-3A was shown, in the present disclosure each of the rest 13 anti-Siglec-3 antibodies also exhibited similar biological activity as that of 4H3-3A.

### 2.1 Size-Exclusion Chromatography

In this example, the anti-Siglec-3 antibody 4H3-3A of Example 1, and two other anti-Siglec-3 antibodies 10C8 and 2B9 were independently subjected to size-exclusion chromatography (data not shown). Note that both the anti-Siglec-3 antibodies 10C8 and 2B9 are known to suppress HBV infection and were included in this example as positive experimental controls.

The anti-Siglec-3 antibody 4H3-3A was capable of being harvested in a single fraction (i.e., fraction 11.66), indicating that it was a single antibody molecule, and the molecular weight was around 150 kDa (data not shown). As to the antibody 10C8, a single fraction (i.e., fraction 9.02) with a significant peak tailing was observed in size-exclusion chromatography, indicating that it was prone to form oligomers, rather than a single antibody molecule (data not shown). Twin peaks (i.e., fractions 11.52 and 16.61) were observed for the antibody 2B9 in size-exclusion chromatography, in which one peak (i.e., fraction 11.52) represented the antibody molecule, while the other peak (i.e., fraction 16.61) represented accompanying proteins, suggesting that the purity of 2B9 was poor than that of the antibody 4H3-3A (data not shown).

### 2.2 Binding Affinity

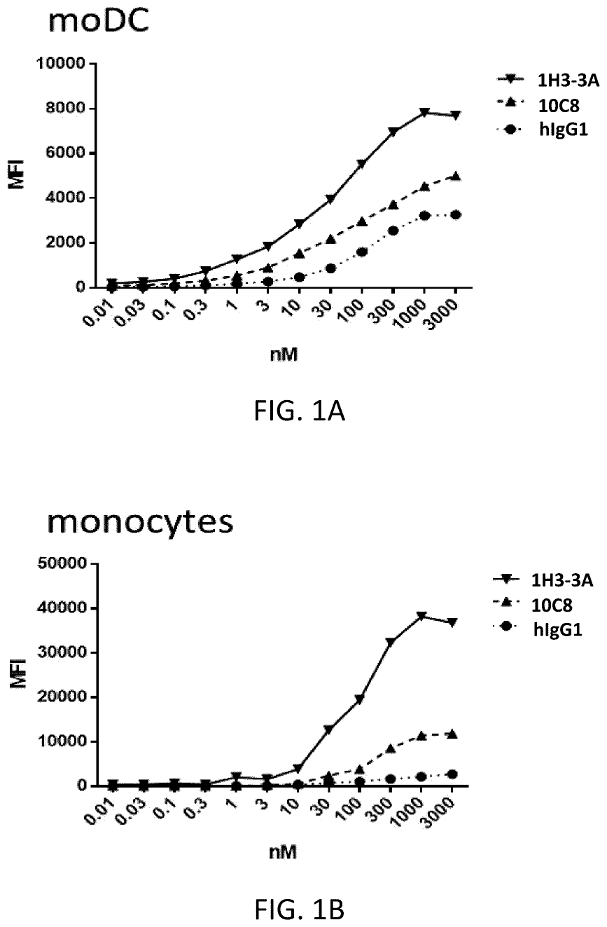

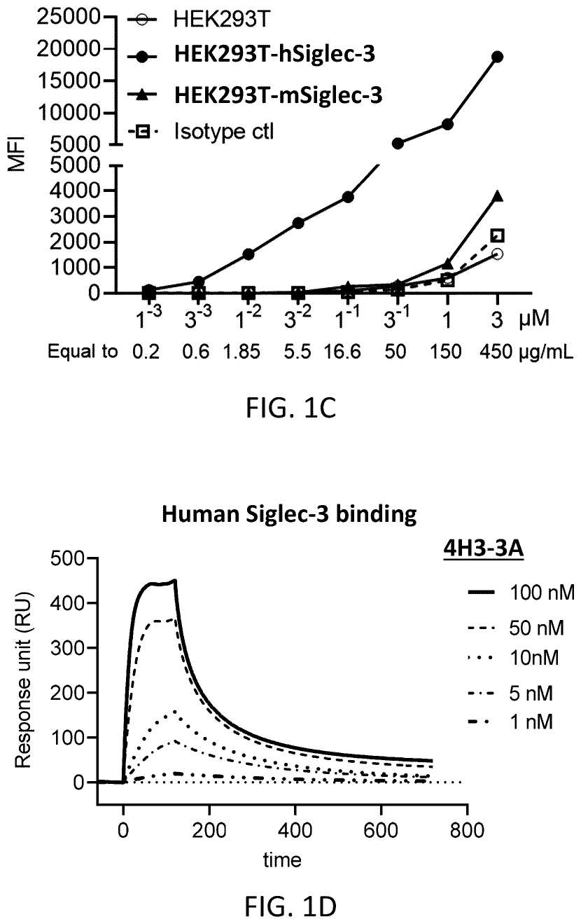

In this example, the optimal concentrations of the antibodies 4H3-3A, 10C8 or 2B9 toward human primary cells (including human moDCs and monocytes, which express endogenous Siglec-3), as well as human cell line HEK-293T expressing exogenous human or mouse Siglec-3, were determined using the well-established antibody titration flow cytometry in accordance with the steps described in the “Materials and Methods” section, and results are presented in FIGS. 1A-1C.

For moDCs, the present antibody 4H3-3A exhibited relatively higher affinity toward moDCs as compared to that of the antibodies 10C8 or hIgG1 (FIG. 1A). Similar trend was also observed in monocytes; moreover, the antibody 4H3-3A also exhibited a much higher selectivity toward monocytes than that of the antibodies 10C8 or hIgG1 (FIG. 1 B). Furthermore, as shown in FIG. 1C, the antibody 4H3-3A exhibited dose-dependent binding to human Siglec-3, but not to mouse Siglec-3, expressed in HEK-293T cells; the antibody 4H3-3A did not cross react to mouse Siglec-3 up to 50 g/ml. The binding affinity of the antibody 4H3-3A to human Siglec-3 was directly measured using Biacore analysis, as shown in FIG. 1D and summarized in Table 1, based on the findings as described above, in which the affinity of the antibody 4H3-3A for human Siglec-3 was determined to be 8.88×10⁻⁹ M, the K_{D} value.

TABLE 1
Details from the Biacore analyses

| Kₐ (1/Ms) | K_{d} (1/s) | K_{D} (M) | Chi² |
|---|---|---|---|
| 4.21 × 10⁶ | 3.73 × 10⁻² | 8.88 × 10⁻⁹ | 72.417 |

Taken together, the present antibody 4H3-3A exhibited greater binding affinity than 10C8 in terms of binding to human endogenous or exogenous Siglec-3.

### Example 3 Effects of the Antibody 4H3-3A on Treating HBV Infection

### 3.1 T Cell Activation and Regulation

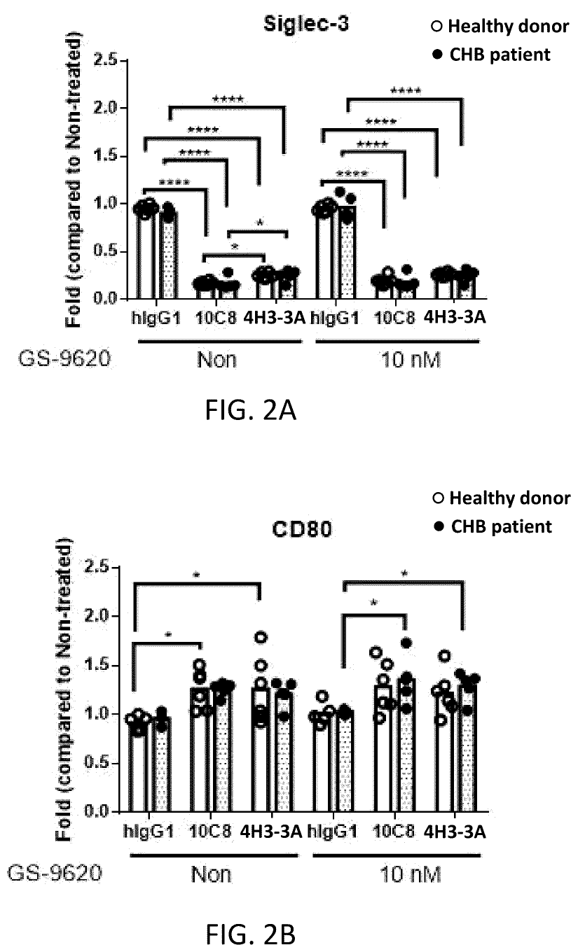

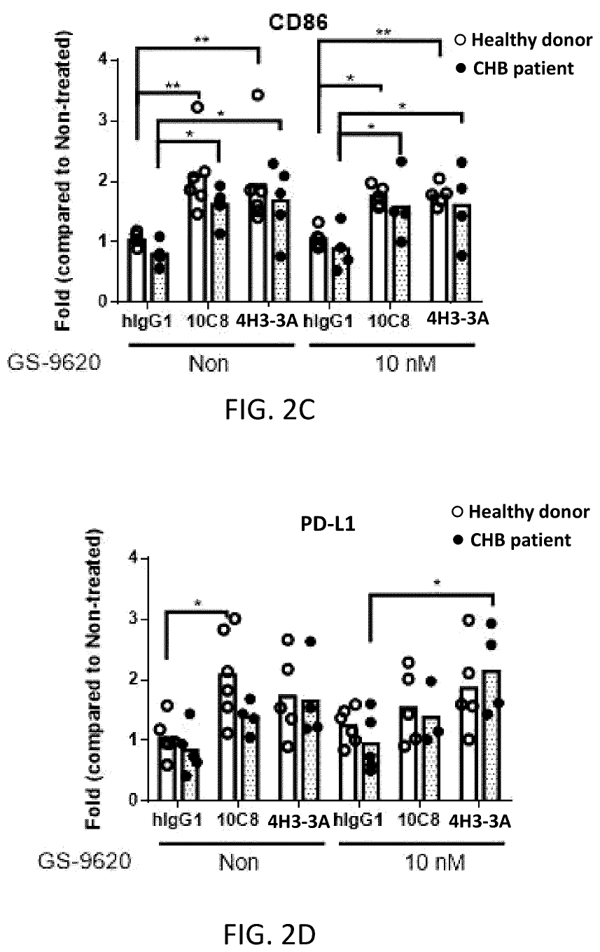

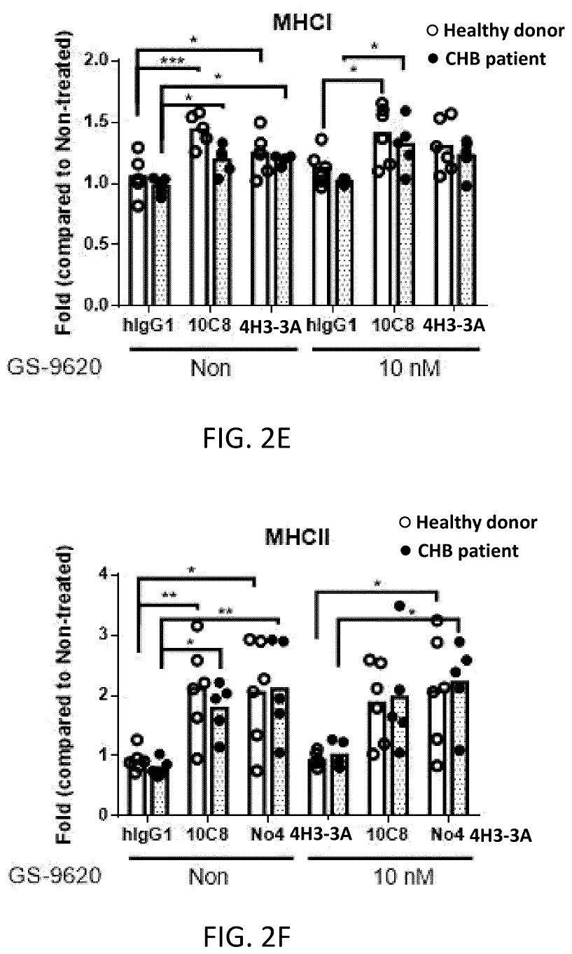

Recent studies showed that HBV may suppress host immune responses via interaction with Siglec-3, thus, application of an anti-Siglec-3 antibody that blocks the interaction between HBV and Siglec-3 may therefore restore the host immune responses. Following this idea, PBMCs from the healthy donors or the CHB patients (as HBV carriers) were treated with the anti-Siglec-3 antibody 4H3-3A, the known antibody 10C8, or control antibody hIgG1, and the immune response of each group of PBMCs was evaluated by monitoring the expressions of Siglec-3, and several other surface markers, which included CD80, CD86, PD-L1, MHC-I, and MHC-II (which were involved in antigen presentation of CD14⁺ cells of PBMCs), using flow cytometry. Further, in the same test, a toll-like receptor-7 (TLR-7) agonist GS-9620 (10 nM), a drug candidate currently being tested in clinical trials forthe treatment of chronic HBV infection, was included in parallel, in order to investigate if the presence of GS-9620 would bring about a synergistic effect on enhancing the host immune responses. Results are provided in FIGS. 2A to 2F.

As shown in FIG. 2A, the expression of Siglec-3 decreased in the CD14⁺ cells collected from the healthy donors or the CHB patients after treatment of the antibodies 10C8 or 4H3-3A. However, the addition of GS-9620 after treatment of the anti-Siglec-3 antibodies led to a slight bounced back of the expression of Siglec-3 (FIG. 2A). By contrast, the expressions of CD80, CD86, PD-L1, MHC-I, and MHC-II increased in the CD14⁺ cells collected from the healthy donors or the CHB patients after treatment of the antibodies 10C8 or 4H3-3A, and the pattern of the expressions of those markers remained similar even in the presence of GS-9620, which is a TLR-7 agonist and antiviral agent (FIGS. 2B-2F).

Taken together, the results confirmed that the anti-Siglec-3 antibody 4H3-3A, alone or in combination with GS-9620, may promote the antigen-presenting ability of CD14⁺ cells in PBMCs.

### 3.2 Antiviral Effect

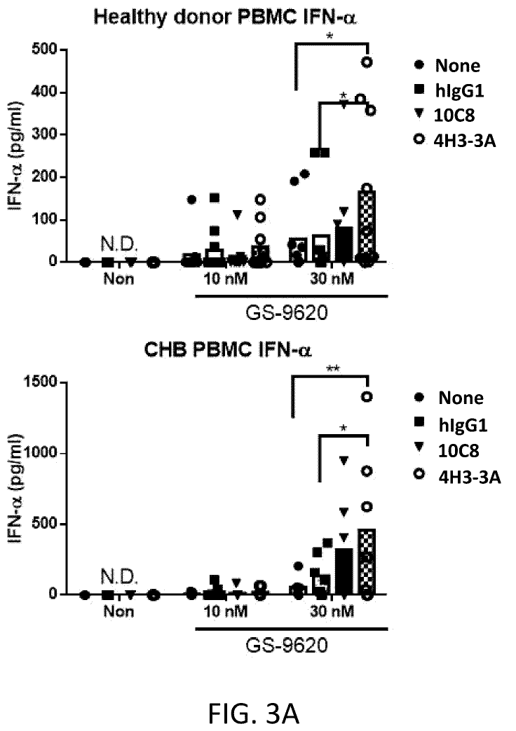

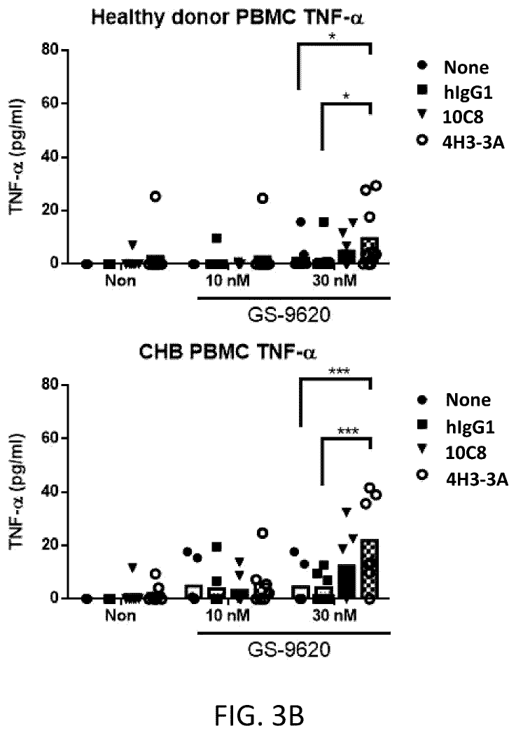

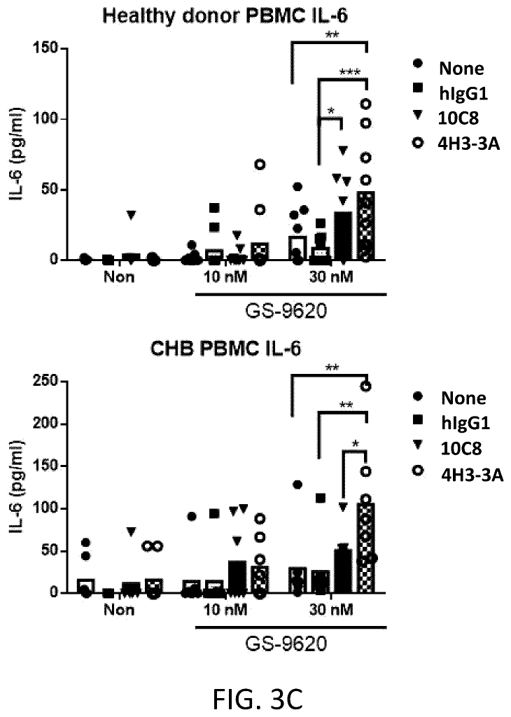

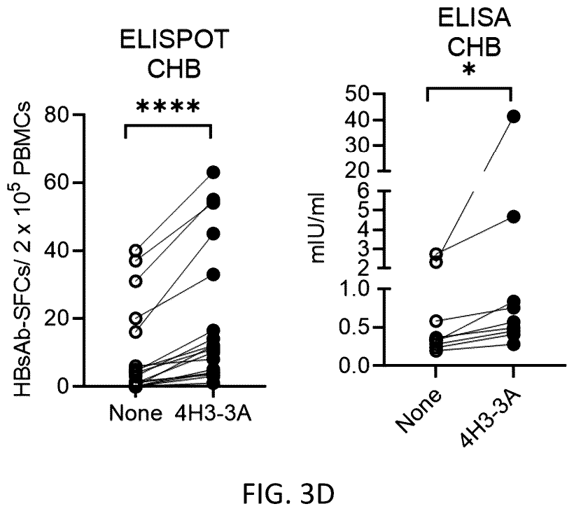

In this example, the ability of the anti-Siglec-3 antibody 4H3-3A in to induce the expressions of antiviral cytokines (including IFN-α, TNF-α, and IL-6) in the absence or presence of GS-9602 was investigated, and the results are provided in FIGS. 3A-3C.

As shown in FIG. 3A, in the absence or low dose (10 nM) of GS-9620, treatment with the anti-Siglec-3 antibodies 4H3-3A or 10C8 did not significantly enhance the expression of IFN-α in the PBMCs of the healthy donors or the CHB patients, while in the presence of high dose (30 nM) of GS-9620, only the anti-Siglec-3 antibody 4H3-3A was capable of enhancing the expression of IFN-α in the PBMCs of the healthy donors or the CHB patients. The results suggested the anti-Siglec-3 antibody 4H3-3A and high concentration of GS-9620 may synergistically induce the secretion of IFN-α, respectively.

Similar results were seen in the induction of TNF-α and IL-6 secretion. References are made to FIGS. 3B and 3C, in which treatment with the anti-Siglec-3 antibodies 4H3-3A or 10C8, with or without low dose (10 nM) of GS-9620, independently induced minimal amounts of TNF-α and IL-6 secretion, while the combined treatment of the anti-Siglec-3 antibody 4H3-3A and high dose (30 nM) of GS-9620 significantly increased the expressions of TNF-α or IL-6 in the PBMCs of the healthy donors or the CHB patients.

In addition, the effect of the antibody 4H3-3A on inducing the production of anti-HBsAg antibodies in the PBMCs of the CHB patients was investigated. As shown in FIG. 3D, both of the ELISpot or the ELISA results demonstrated that the production of anti-HBsAg antibodies in the PBMCs of CHB patients was found to be triggered by the antibody 4H3-3A, indicating that the antibody 4H3-3A indeed possesses an effective ability to eliminate HBV infection.

Taken together, these results demonstrated that as compared to the antibody 10C8, the anti-Siglec-3 antibody 4H3-3A has a greater ability to induce secretion of antiviral cytokines (including IFN-α, TNF-α, and IL-6) in CD14⁺ cells of PBMCs, and a synergistical enhanced secretion existed when the anti-Siglec-3 antibody 4H3-3A was administered together with the use of GS-9620. Further, the antibody 4H3-3A may directly increase the level of anti-HBsAg antibodies.

### Example 4 Effects of the Antibody 4H3-3A on Treating Alzheimer's Disease

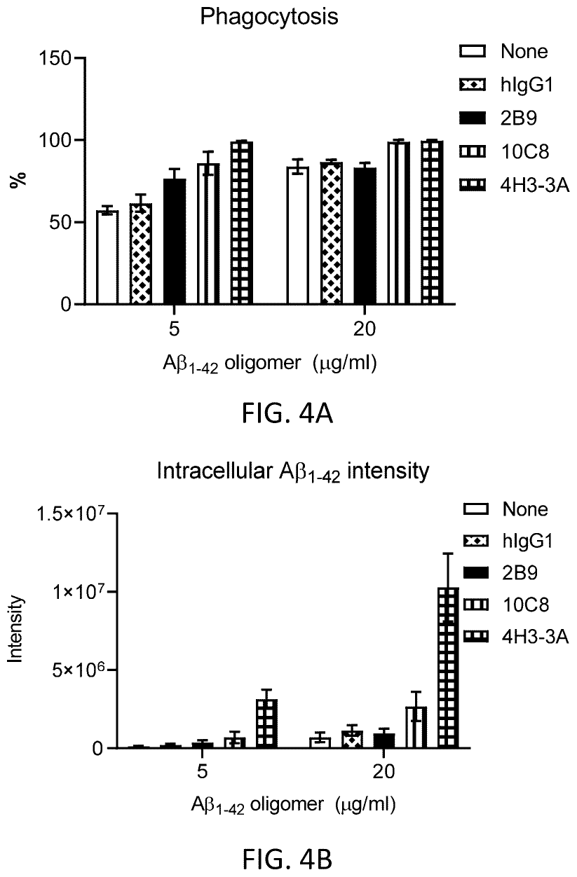

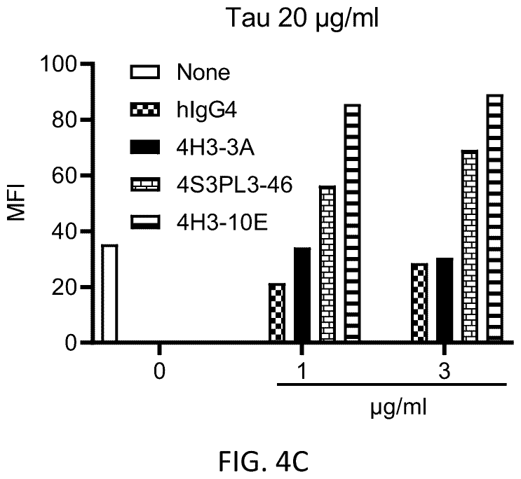

In the present example, the efficacy of the antibody 4H3-3A in treating Alzheimer's disease was investigated. The main pathological manifestation of Alzheimer's disease is the accumulation of disease-associated proteins, including Aβ₁₋₄₂ and Tau, in microglia. Phagocytosis of these mutant proteins by microglia is an important cellular process for eliminating them and treating Alzheimer's disease. It was found that the antibody 4H3-3A promoted the phargocytosis of Aβ₁₋₄₂ by microglia, as evidenced by both the percentage rate (FIG. 4A) and the intensity (FIG. 4B) of the Aβ₁₋₄₂ phargocytosis. Similar results were found in Tau phargocytosis, as the antibody 4H3-3A, as well as two other anti-Siglec-3 antibodies 4S3PL3-46 and 4H3-10E, promoted the phargocytosis of Tau by microglia (FIG. 4C). Collectively, these data evidenced that the antibody 4H3-3A can be used in treating Alzheimer's disease.

### Example 5 Effects of the Antibody 4H3-3A on Treating SLE

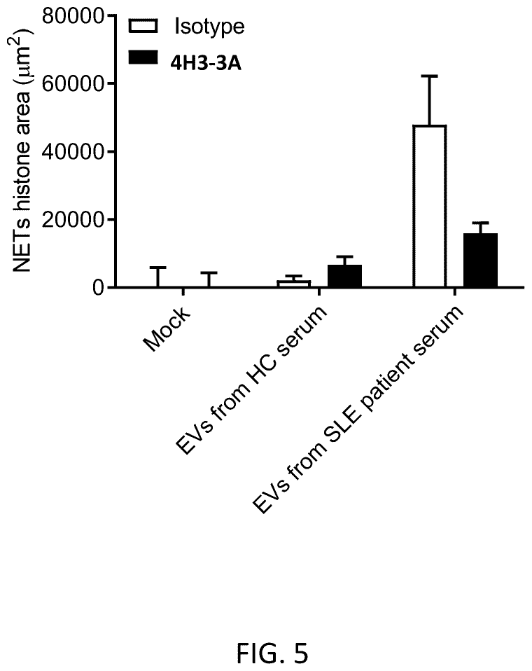

Further, the potential of the antibody 4H3-3A to treat autoimmune diseases, specifically SLE, as an exemplary autoimmune disease representative, was investigated. As provided in FIG. 5, the antibody 4H3-3A specifically suppressed the formation of NETs, a hallmark of inflammation, in EVs derived from SLE patients' serum, but not in those derived from healthy donors' serum. As such, the antibody 4H3-3A possesses the ability to suppress inflammation, so as to treat SLE.

In sum, the present disclosure has shown that the anti-Siglec-3 antibody presented herein has a high affinity to human Siglec-3, and has the potential to modulate an individual's immune response to combat HBV infection. The anti-Siglec-3 antibody presented herein also has the potential to eliminate disease-associated proteins in microglia and suppress inflammation, as well as suppress tumor growth. All the features evidence that the present anti-Siglec-3 antibody is a useful therapeutic antibody that may prevent or treat HBV infection, neurodegenerative diseases, autoimmune diseases, and cancer.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A recombinant antibody comprising a light chain variable (VL) region and a heavy chain variable (VH) region, wherein the VL region comprises a first complementarity determining region (CDR-L1), a second CDR (CDR-L2), and a third CDR (CDR-L3), and the VH region comprises a first complementarity determining region (CDR-H1), a second CDR (CDR-H2), and a third CDR (CDR-H3), wherein
the CDR-L1 is selected from the group consisting of SEQ ID NOs: 1 and 57;
the CDR-L2 is selected from the group consisting of SEQ ID NOs: 2 and 58;
the CDR-L3 is selected from the group consisting of SEQ ID NOs: 3 and 59;
the CDR-H1 is selected from the group consisting of SEQ ID NOs: 5, 9, 13, 17, 21, 25, 29, 33, 37, 41, 45, 49, 53, and 61;
the CDR-H2 is selected from the group consisting of SEQ ID NOs: 6, 10, 14, 18, 22, 26, 30, 34, 38, 42, 46, 50, 54, and 62; and
the CDR-H3 is selected from the group consisting of SEQ ID NOs: 7, 11, 15, 19, 23, 27, 31, 35, 39, 43, 47, 51, 55, and 63.

2. The recombinant antibody of claim 1, wherein
the CDR-L1 to CDR-L3 and the CDR-H1 to CDR-H3 respectively comprise the amino acid sequences of SEQ ID NOs: 1-3 and SEQ ID NOs: 5-7;
the CDR-L1 to CDR-L3 and the CDR-H1 to CDR-H3 respectively comprise the amino acid sequences of SEQ ID NOs: 1-3 and SEQ ID NOs: 9-11;
the CDR-L1 to CDR-L3 and the CDR-H1 to CDR-H3 respectively comprise the amino acid sequences of SEQ ID NOs: 1-3 and SEQ ID NOs: 13-15;
the CDR-L1 to CDR-L3 and the CDR-H1 to CDR-H3 respectively comprise the amino acid sequences of SEQ ID NOs: 1-3 and SEQ ID NOs: 17-19;
the CDR-L1 to CDR-L3 and the CDR-H1 to CDR-H3 respectively comprise the amino acid sequences of SEQ ID NOs: 1-3 and SEQ ID NOs: 21-23;
the CDR-L1 to CDR-L3 and the CDR-H1 to CDR-H3 respectively comprise the amino acid sequences of SEQ ID NOs: 1-3 and SEQ ID NOs: 25-27;
the CDR-L1 to CDR-L3 and the CDR-H1 to CDR-H3 respectively comprise the amino acid sequences of SEQ ID NOs: 1-3 and SEQ ID NOs: 29-31;
the CDR-L1 to CDR-L3 and the CDR-H1 to CDR-H3 respectively comprise the amino acid sequences of SEQ ID NOs: 1-3 and SEQ ID NOs: 33-35;
the CDR-L1 to CDR-L3 and the CDR-H1 to CDR-H3 respectively comprise the amino acid sequences of SEQ ID NOs: 1-3 and SEQ ID NOs: 37-39;
the CDR-L1 to CDR-L3 and the CDR-H1 to CDR-H3 respectively comprise the amino acid sequences of SEQ ID NOs: 1-3 and SEQ ID NOs: 41-43;
the CDR-L1 to CDR-L3 and the CDR-H1 to CDR-H3 respectively comprise the amino acid sequences of SEQ ID NOs: 1-3 and SEQ ID NOs: 45-47;
the CDR-L1 to CDR-L3 and the CDR-H1 to CDR-H3 respectively comprise the amino acid sequences of SEQ ID NOs: 1-3 and SEQ ID NOs: 49-51;
the CDR-L1 to CDR-L3 and the CDR-H1 to CDR-H3 respectively comprise the amino acid sequences of SEQ ID NOs: 1-3 and SEQ ID NOs: 53-55; or
the CDR-L1 to CDR-L3 and the CDR-H1 to CDR-H3 respectively comprise the amino acid sequences of SEQ ID NOs: 57-59 and SEQ ID NOs: 61-63.

3. The recombinant antibody of claim 2, wherein
the VL and VH regions respectively comprise an amino acid sequence at least 85% identical to SEQ ID NOs: 4 and 8;
the VL and VH regions respectively comprise an amino acid sequence at least 85% identical to SEQ ID NOs: 4 and 12;
the VL and VH regions respectively comprise an amino acid sequence at least 85% identical to SEQ ID NOs: 4 and 16;
the VL and VH regions respectively comprise an amino acid sequence at least 85% identical to SEQ ID NOs: 4 and 20;
the VL and VH regions respectively comprise an amino acid sequence at least 85% identical to SEQ ID NOs: 4 and 24;
the VL and VH regions respectively comprise an amino acid sequence at least 85% identical to SEQ ID NOs: 4 and 28;
the VL and VH regions respectively comprise an amino acid sequence at least 85% identical to SEQ ID NOs: 4 and 32;
the VL and VH regions respectively comprise an amino acid sequence at least 85% identical to SEQ ID NOs: 4 and 36;
the VL and VH regions respectively comprise an amino acid sequence at least 85% identical to SEQ ID NOs: 4 and 40;
the VL and VH regions respectively comprise an amino acid sequence at least 85% identical to SEQ ID NOs: 4 and 44;
the VL and VH regions respectively comprise an amino acid sequence at least 85% identical to SEQ ID NOs: 4 and 48;
the VL and VH regions respectively comprise an amino acid sequence at least 85% identical to SEQ ID NOs: 4 and 52;
the VL and VH regions respectively comprise an amino acid sequence at least 85% identical to SEQ ID NOs: 4 and 56; or
the VL and VH regions respectively comprise an amino acid sequence at least 85% identical to SEQ ID NOs: 60 and 64.

4. The recombinant antibody of claim 3, wherein
the VL and VH regions respectively comprise the amino acid sequences of SEQ ID NOs: 4 and 8;
the VL and VH regions respectively comprise the amino acid sequences of SEQ ID NOs: 4 and 12;
the VL and VH regions respectively comprise the amino acid sequences of SEQ ID NOs: 4 and 16;
the VL and VH regions respectively comprise the amino acid sequences of SEQ ID NOs: 4 and 20;
the VL and VH regions respectively comprise the amino acid sequences of SEQ ID NOs: 4 and 24;
the VL and VH regions respectively comprise the amino acid sequences of SEQ ID NOs: 4 and 28;
the VL and VH regions respectively comprise the amino acid sequences of SEQ ID NOs: 4 and 32;
the VL and VH regions respectively comprise the amino acid sequences of SEQ ID NOs: 4 and 36;
the VL and VH regions respectively comprise the amino acid sequences of SEQ ID NOs: 4 and 40;
the VL and VH regions respectively comprise the amino acid sequences of SEQ ID NOs: 4 and 44;
the VL and VH regions respectively comprise the amino acid sequences of SEQ ID NOs: 4 and 48;
the VL and VH regions respectively comprise the amino acid sequences of SEQ ID NOs: 4 and 52;
the VL and VH regions respectively comprise the amino acid sequences of SEQ ID NOs: 4 and 56; or
the VL and VH regions respectively comprise the amino acid sequences of SEQ ID NOs: 60 and 64.

5. A pharmaceutical composition comprising an effective amount of the recombinant antibody of claim 1, and a pharmaceutically acceptable excipient.

6. The pharmaceutical composition of claim 5, wherein in the recombinant antibody,
the CDR-L1 to CDR-L3 and the CDR-H1 to CDR-H3 respectively comprise the amino acid sequences of SEQ ID NOs: 1-3 and SEQ ID NOs: 5-7;
the CDR-L1 to CDR-L3 and the CDR-H1 to CDR-H3 respectively comprise the amino acid sequences of SEQ ID NOs: 1-3 and SEQ ID NOs: 9-11;
the CDR-L1 to CDR-L3 and the CDR-H1 to CDR-H3 respectively comprise the amino acid sequences of SEQ ID NOs: 1-3 and SEQ ID NOs: 13-15;
the CDR-L1 to CDR-L3 and the CDR-H1 to CDR-H3 respectively comprise the amino acid sequences of SEQ ID NOs: 1-3 and SEQ ID NOs: 17-19;
the CDR-L1 to CDR-L3 and the CDR-H1 to CDR-H3 respectively comprise the amino acid sequences of SEQ ID NOs: 1-3 and SEQ ID NOs: 21-23;
the CDR-L1 to CDR-L3 and the CDR-H1 to CDR-H3 respectively comprise the amino acid sequences of SEQ ID NOs: 1-3 and SEQ ID NOs: 25-27;
the CDR-L1 to CDR-L3 and the CDR-H1 to CDR-H3 respectively comprise the amino acid sequences of SEQ ID NOs: 1-3 and SEQ ID NOs: 29-31;
the CDR-L1 to CDR-L3 and the CDR-H1 to CDR-H3 respectively comprise the amino acid sequences of SEQ ID NOs: 1-3 and SEQ ID NOs: 33-35;
the CDR-L1 to CDR-L3 and the CDR-H1 to CDR-H3 respectively comprise the amino acid sequences of SEQ ID NOs: 1-3 and SEQ ID NOs: 37-39;
the CDR-L1 to CDR-L3 and the CDR-H1 to CDR-H3 respectively comprise the amino acid sequences of SEQ ID NOs: 1-3 and SEQ ID NOs: 41-43;
the CDR-L1 to CDR-L3 and the CDR-H1 to CDR-H3 respectively comprise the amino acid sequences of SEQ ID NOs: 1-3 and SEQ ID NOs: 45-47;
the CDR-L1 to CDR-L3 and the CDR-H1 to CDR-H3 respectively comprise the amino acid sequences of SEQ ID NOs: 1-3 and SEQ ID NOs: 49-51;
the CDR-L1 to CDR-L3 and the CDR-H1 to CDR-H3 respectively comprise the amino acid sequences of SEQ ID NOs: 1-3 and SEQ ID NOs: 53-55; or
the CDR-L1 to CDR-L3 and the CDR-H1 to CDR-H3 respectively comprise the amino acid sequences of SEQ ID NOs: 57-59 and SEQ ID NOs: 61-63.

7. The pharmaceutical composition of claim 6, wherein in the recombinant antibody,
the VL and VH regions respectively comprise an amino acid sequence at least 85% identical to SEQ ID NOs: 4 and 8;
the VL and VH regions respectively comprise an amino acid sequence at least 85% identical to SEQ ID NOs: 4 and 12;
the VL and VH regions respectively comprise an amino acid sequence at least 85% identical to SEQ ID NOs: 4 and 16;
the VL and VH regions respectively comprise an amino acid sequence at least 85% identical to SEQ ID NOs: 4 and 20;
the VL and VH regions respectively comprise an amino acid sequence at least 85% identical to SEQ ID NOs: 4 and 24;
the VL and VH regions respectively comprise an amino acid sequence at least 85% identical to SEQ ID NOs: 4 and 28;
the VL and VH regions respectively comprise an amino acid sequence at least 85% identical to SEQ ID NOs: 4 and 32;
the VL and VH regions respectively comprise an amino acid sequence at least 85% identical to SEQ ID NOs: 4 and 36;
the VL and VH regions respectively comprise an amino acid sequence at least 85% identical to SEQ ID NOs: 4 and 40;
the VL and VH regions respectively comprise an amino acid sequence at least 85% identical to SEQ ID NOs: 4 and 44;
the VL and VH regions respectively comprise an amino acid sequence at least 85% identical to SEQ ID NOs: 4 and 48;
the VL and VH regions respectively comprise an amino acid sequence at least 85% identical to SEQ ID NOs: 4 and 52;
the VL and VH regions respectively comprise an amino acid sequence at least 85% identical to SEQ ID NOs: 4 and 56; or
the VL and VH regions respectively comprise an amino acid sequence at least 85% identical to SEQ ID NOs: 60 and 64.

8. The pharmaceutical composition of claim 7, wherein in the recombinant antibody,
the VL and VH regions respectively comprise the amino acid sequences of SEQ ID NOs: 4 and 8;
the VL and VH regions respectively comprise the amino acid sequences of SEQ ID NOs: 4 and 12;
the VL and VH regions respectively comprise the amino acid sequences of SEQ ID NOs: 4 and 16;
the VL and VH regions respectively comprise the amino acid sequences of SEQ ID NOs: 4 and 20;
the VL and VH regions respectively comprise the amino acid sequences of SEQ ID NOs: 4 and 24;
the VL and VH regions respectively comprise the amino acid sequences of SEQ ID NOs: 4 and 28;
the VL and VH regions respectively comprise the amino acid sequences of SEQ ID NOs: 4 and 32;
the VL and VH regions respectively comprise the amino acid sequences of SEQ ID NOs: 4 and 36;
the VL and VH regions respectively comprise the amino acid sequences of SEQ ID NOs: 4 and 40;
the VL and VH regions respectively comprise the amino acid sequences of SEQ ID NOs: 4 and 44;
the VL and VH regions respectively comprise the amino acid sequences of SEQ ID NOs: 4 and 48;
the VL and VH regions respectively comprise the amino acid sequences of SEQ ID NOs: 4 and 52;
the VL and VH regions respectively comprise the amino acid sequences of SEQ ID NOs: 4 and 56; or
the VL and VH regions respectively comprise the amino acid sequences of SEQ ID NOs: 60 and 64.

9. A method for preventing and/or treating hepatitis B virus (HBV) infection, a neurodegenerative disease, or an autoimmune disease in a subject, comprising administering to the subject an effective amount of the pharmaceutical composition of claim 5.

10. The method of claim 9, wherein the subject has the HBV infection.

11. The method of claim 10, further comprising administering to the subject an effective amount of an antiviral agent.

12. The method of claim 11, wherein the antiviral agent is selected from the group consisting of acyclovir, adefovir, brincidofovir, brivudine, cidofovir, clevudine, cytarabine, docosanol, edoxudine, entecavir, famciclovir, filociclovir, fomivirsen, foscarnet, ganciclovir, idoxuridine, imiquimod, interferon-α, lamivudine, lobucavir, maribavir, methisazone, moroxydine, penciclovir, peginterferon-α, podophyllotoxin, ribavirin, rifampicin, resiquimod, sorivudine, taribavirin, tecovirimat, telbivudine, tenofovir, thymosin-α, trifluridine, tromantadine, valaciclovir, valganciclovir, vesatolimod (GS-9620), vidarabine, viread, and a combination thereof.

13. The method of claim 12, wherein the antiviral agent is vesatolimod (GS-9620).

14. The method of claim 9, wherein the subject has the neurodegenerative disease.

15. The method of claim 14, wherein the neurodegenerative disease is selected from the group consisting of Alzheimer's disease, Batten disease, Parkinson's disease, dementia with Lewy bodies, multiple system atrophy, bovine spongiform encephalopathy, Creutzfeldt-Jakob disease, Huntington's disease, amyotrophic lateral sclerosis, ataxia telangiectasia, multiple sclerosis, spinocerebellar atrophy, HIV-associated neurocognitive disorders, Pick's disease, Krabbe's disease, spinal and bulbar muscular atrophy, primary lateral sclerosis, Cockayne syndrome, spinal muscular atrophy, tabes dorsalis, progressive supranuclear palsy, and Pelizaeus-Merzbacher disease.

16. The method of claim 14, further comprising administering to the subject an effective amount of acyclovir, aducanumab, amantadine, apomorphine, baclofen, carbidopa, carbidopa, dantrolene, donepezil, entacapone, foscarnet, galantamine, levodopa, memantine, penciclovir, pramipexole, rasagiline, riluzole, rivastigmine, ropinirole, selegiline, tacrine, tetrabenazine, tizanidine, or tolcapone.

17. The method of claim 9, wherein the subject has the autoimmune disease.

18. The method of claim 17, wherein the autoimmune disease is selected from the group consisting of acute disseminated encephalomyelitis, Addison's disease, alopecia areata, antiphospholipid syndrome, antisynthetase syndrome, asthma, autoimmune angioedema, autoimmune hepatitis, autoimmune pancreatitis, autoimmune polyendocrine syndrome type 1-3, autoimmune progesterone dermatitis, autoimmune thyroiditis, autoimmune urticaria, bullous pemphigoid, chronic hives, cicatricial pemphigoid, coeliac disease, Crohn's disease, dermatitis herpetiformis, dermatomyositis, diabetes mellitus type 1, discoid lupus erythematosus, endometriosis, epidermolysis bullosa acquisita, esophageal achalasia, erythema nodosum, familial mediterranean fever, gestational pemphigoid, Graves' disease, gout, hidradenitis suppurativa, IgA vasculitis, inflammatory bowel disease, interstitial cystitis, lichen planus, lichen sclerosus, lupus nephritis, morphea, Mucha-Habermann disease, Muckle-Wells syndrome, myocarditis, myasthenia gravis, pemphigus vulgaris, pityriasis lichenoides et varioliformis acuta, postmyocardial infarction syndrome, primary biliary cholangitis, primary sclerosing cholangitis, psoriasis, rheumatoid arthritis, Sjögren syndrome, subacute bacterial endocarditis, systemic lupus erythematosus, systemic scleroderma, ulcerative colitis, and vitiligo.

19. The method of claim 17, further comprising administering to the subject an effective amount of abatacept, acitretin, adalimumab, alefacept, anakinra, anthralin, apremilast, azathioprine, baricitinib, belimumab, benralizumab, betamethasone, bimekizumab, brazikumab, brodalumab, calcipotriene, calcipotriol, calcitriol, canakinumab, certolizumab, clobetasol, coal tar, colchicine, cyclosporine, dapsone, dithranol, dexamethasone, dupilumab, eculizumab, etanercept, fluocinolone, golimumab, guselkumab, hydroxychloroquine, hydrocortisone, infliximab, ixekizumab, lenercept, mepolizumab, methotrexate, methylprednisolone, mirikizumab, mycophenolate mofetil, omalizumab, prednisone, perakizumab, pimecrolimus, remtolumab, reslizumab, rilonacept, risankizumab, rituximab, sarilumab, secukinumab, sulfasalazine, tacrolimus, tazarotene, tildrakizumab, tocilizumab, tofacitinib, tretinoin, upadacitinib, ustekinumab, vedolizumab, or vunakizumab.

20-22. (canceled)


SEQUENCE LISTING (127 sequences)

SEQ ID NO: 1 - protein, 4 aa, synthetic construct
VYGY

SEQ ID NO: 2 - protein, 7 aa, synthetic construct
LISYTPG

SEQ ID NO: 3 - protein, 7 aa, synthetic construct
QYYDFPI

SEQ ID NO: 4 - protein, 113 aa, synthetic construct
DGTDIQMTQSPSSLSASVGDRVTITCRASQDVYGYVAWYQQKPGKAPKLLISYTPGLYSGVPSRFSGSGSGTDFTLTISSLQPEDFATYYCQQYYDFPITFGQGTKVEIKRTV

SEQ ID NO: 5 - protein, 6 aa, synthetic construct
TIDDGF

SEQ ID NO: 6 - protein, 10 aa, synthetic construct
GIGPYGGYTY

SEQ ID NO: 7 - protein, 11 aa, synthetic construct
ARFNDHWDLWM

SEQ ID NO: 8 - protein, 124 aa, synthetic construct
VQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFNDHWDLWMDYWGQGTLVTVSSASTKG

SEQ ID NO: 9 - protein, 6 aa, synthetic construct
TIDDGF

SEQ ID NO: 10 - protein, 10 aa, synthetic construct
GIGPYGGYTY

SEQ ID NO: 11 - protein, 11 aa, synthetic construct
ARFRDDKPQMM

SEQ ID NO: 12 - protein, 124 aa, synthetic construct
VQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFRDDKPQMMDYWGQGTLVTVSSASTKG

SEQ ID NO: 13 - protein, 6 aa, synthetic construct
TIDDGF

SEQ ID NO: 14 - protein, 10 aa, synthetic construct
GIGPYGGYTY

SEQ ID NO: 15 - protein, 11 aa, synthetic construct
ARFRDTQSQTM

SEQ ID NO: 16 - protein, 124 aa, synthetic construct
VQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFRDTQSQTMDYWGQGTLVTVSSASTKG

SEQ ID NO: 17 - protein, 6 aa, synthetic construct
TIDDGF

SEQ ID NO: 18 - protein, 10 aa, synthetic construct
GIGPYGGYTY

SEQ ID NO: 19 - protein, 11 aa, synthetic construct
ARFGHPPYDYM

SEQ ID NO: 20 - protein, 124 aa, synthetic construct
VQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFGHPPYDYMDYWGQGTLVTVSSASTKG

SEQ ID NO: 21 - protein, 6 aa, synthetic construct
TIDDGF

SEQ ID NO: 22 - protein, 10 aa, synthetic construct
GIGPYGGYTY

SEQ ID NO: 23 - protein, 11 aa, synthetic construct
ARFQNSPTKTM

SEQ ID NO: 24 - protein, 124 aa, synthetic construct
VQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFQNSPTKTMDYWGQGTLVTVSSASTKG

SEQ ID NO: 25 - protein, 6 aa, synthetic construct
TIDDGF

SEQ ID NO: 26 - protein, 10 aa, synthetic construct
GIGPYGGYTY

SEQ ID NO: 27 - protein, 11 aa, synthetic construct
ARFATHDQPLM

SEQ ID NO: 28 - protein, 124 aa, synthetic construct
VQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFATHDQPLMDYWGQGTLVTVSSASTKG

SEQ ID NO: 29 - protein, 6 aa, synthetic construct
TIHGSW

SEQ ID NO: 30 - protein, 10 aa, synthetic construct
GIGPYGGYTY

SEQ ID NO: 31 - protein, 11 aa, synthetic construct
ARFNDHQDLWM

SEQ ID NO: 32 - protein, 124 aa, synthetic construct
VQLVESGGGLVQPGGSLRLSCAASGFTIHGSWIHWVRQAPGKGLEWVAGIGPYGGYTYYADSVKGRFTISADTSKNTAYLQMNSLRAEDTAVYYCARFNDHQDLWMDYWGQGTLVTVSSASTKG

SEQ ID NO: 33 - protein, 6 aa, synthetic construct
TIHGSW

SEQ ID NO: 34 - protein, 10 aa, synthetic construct
GIGPYGGYTY

SEQ ID NO: 35 - protein, 11 aa, synthetic construct
ARFNDHWDLWM

SEQ ID NO: 36 - protein, 124 aa, synthetic construct
VQLVESGGGLVQPGGSLRLSCAASGFTIHGSWIHWVRQAPGKGLEWVAGIGPYGGYTYYADSVKGRFTISADTSKNTAYLQMNSLRAEDTAVYYCARFNDHWDLWMDYWGQGTLVTVSSASTKG

SEQ ID NO: 37 - protein, 6 aa, synthetic construct
TISSSY

SEQ ID NO: 38 - protein, 10 aa, synthetic construct
GIGPYGGYTY

SEQ ID NO: 39 - protein, 11 aa, synthetic construct
ARFNDHWDLWM

SEQ ID NO: 40 - protein, 124 aa, synthetic construct
VQLVESGGGLVQPGGSLRLSCAASGFTISSSYIHWVRQAPGKGLEWVAGIGPYGGYTYYADSVKGRFTISADTSKNTAYLQMNSLRAEDTAVYYCARFNDHWDLWMDYWGQGTLVTVSSASTKG

SEQ ID NO: 41 - protein, 6 aa, synthetic construct
TIGSGW

SEQ ID NO: 42 - protein, 10 aa, synthetic construct
GIGPYGGYTY

SEQ ID NO: 43 - protein, 11 aa, synthetic construct
ARFNDHWDLWM

SEQ ID NO: 44 - protein, 124 aa, synthetic construct
VQLVESGGGLVQPGGSLRLSCAASGFTIGSGWIHWVRQAPGKGLEWVAGIGPYGGYTYYADSVKGRFTISADTSKNTAYLQMNSLRAEDTAVYYCARFNDHWDLWMDYWGQGTLVTVSSASTKG

SEQ ID NO: 45 - protein, 6 aa, synthetic construct
TIDDGF

SEQ ID NO: 46 - protein, 10 aa, synthetic construct
AIKPRQGDTS

SEQ ID NO: 47 - protein, 11 aa, synthetic construct
ARFNDHWDLWM

SEQ ID NO: 48 - protein, 124 aa, synthetic construct
VQLVESGGGLVQPGGSLRLSCAASGFTIDDGFIHWVRQAPGKGLEWVAAIKPRQGDTSYADSVKGRFTISADTSKNTAYLQMNSLRAEDTAVYYCARFNDHWDLWMDYWGQGTLVTVSSASTKG

SEQ ID NO: 49 - protein, 6 aa, synthetic construct
TIDDGF

SEQ ID NO: 50 - protein, 10 aa, synthetic construct
AIPPLQGKTY

SEQ ID NO: 51 - protein, 11 aa, synthetic construct
ARFNDHWDLWM

SEQ ID NO: 52 - protein, 124 aa, synthetic construct
VQLVESGGGLVQPGGSLRLSCAASGFTIDDGFIHWVRQAPGKGLEWVAAIPPLQGKTYYADSVKGRFTISADTSKNTAYLQMNSLRAEDTAVYYCARFNDHWDLWMDYWGQGTLVTVSSASTKG

SEQ ID NO: 53 - protein, 6 aa, synthetic construct
TIDDGF

SEQ ID NO: 54 - protein, 10 aa, synthetic construct
TISPNTGHTN

SEQ ID NO: 55 - protein, 11 aa, synthetic construct
ARFNDHWDLWM

SEQ ID NO: 56 - protein, 124 aa, synthetic construct
VQLVESGGGLVQPGGSLRLSCAASGFTIDDGFIHWVRQAPGKGLEWVATISPNTGHTNYADSVKGRFTISADTSKNTAYLQMNSLRAEDTAVYYCARFNDHWDLWMDYWGQGTLVTVSSASTKG

SEQ ID NO: 57 - protein, 4 aa, synthetic construct
VYGY

SEQ ID NO: 58 - protein, 7 aa, synthetic construct
LISYTPG

SEQ ID NO: 59 - protein, 7 aa, synthetic construct
QYSRMPI

SEQ ID NO: 60 - protein, 113 aa, synthetic construct
DGTDIQMTQSPSSLSASVGDRVTITCRASQDVYGYVAWYQQKPGKAPKLLISYTPGLYSGVPSRFSGSGSGTDFTLTISSLQPEDFATYYCQQYSRMPITFGQGTKVEIKRTV

SEQ ID NO: 61 - protein, 6 aa, synthetic construct
TIDDGF

SEQ ID NO: 62 - protein, 10 aa, synthetic construct
GIGPYGGYTY

SEQ ID NO: 63 - protein, 11 aa, synthetic construct
ARFNDHWDLWM

SEQ ID NO: 64 - protein, 124 aa, synthetic construct; identical to SEQ ID NO: 8

SEQ ID NO: 65 - protein, 104 aa, synthetic construct
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 324 aa, synthetic construct
PSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 322 aa, synthetic construct
PSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 237 aa, synthetic construct
MRVPAQLLGLLLLWLPGARCDGTDIQMTQSPSSLSASVGDRVTITCRASQDVYGYVAWYQQKPGKAPKLLISYTPGLYSGVPSRFSGSGSGTDFTLTISSLQPEDFATYYCQQYYDFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 468 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 466 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 468 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFRDDKPQMMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 466 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFRDDKPQMMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 469 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFRDTQSQTMDYWGQGTLVTVSSASTKGPPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 466 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFRDTQSQTMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 469 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFGHPPYDYMDYWGQGTLVTVSSASTKGPPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 466 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFGHPPYDYMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 468 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFQNSPTKTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 466 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFQNSPTKTMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 468 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFATHDQPLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 466 aa, synthetic construct
MGWSLILLFLVAVATRVASEVQLVESGGGLVQPGGSLRLSCAASGFTIDDGFIHWVRQAPGKGLEWVAGIGPYGGYTYYADSVKGRFTISADTSKNTAYLQMNSLRAEDTAVYYCARFATHDQPLMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 468 aa, synthetic construct
MGWSLILLFLVAVATRVASEVQLVESGGGLVQPGGSLRLSCAASGFTIHGSWIHWVRQAPGKGLEWVAGIGPYGGYTYYADSVKGRFTISADTSKNTAYLQMNSLRAEDTAVYYCARFNDHQDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 466 aa, synthetic construct
MGWSLILLFLVAVATRVASEVQLVESGGGLVQPGGSLRLSCAASGFTIHGSWIHWVRQAPGKGLEWVAGIGPYGGYTYYADSVKGRFTISADTSKNTAYLQMNSLRAEDTAVYYCARFNDHQDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 468 aa, synthetic construct
MGWSLILLFLVAVATRVASEVQLVESGGGLVQPGGSLRLSCAASGFTIHGSWIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 466 aa, synthetic construct
MGWSLILLFLVAVATRVASEVQLVESGGGLVQPGGSLRLSCAASGFTIHGSWIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 468 aa, synthetic construct
MGWSLILLFLVAVATRVASEVQLVESGGGLVQPGGSLRLSCAASGFTISSSYIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 466 aa, synthetic construct
MGWSLILLFLVAVATRVASEVQLVESGGGLVQPGGSLRLSCAASGFTISSSYIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 468 aa, synthetic construct
MGWSLILLFLVAVATRVASEVQLVESGGGLVQPGGSLRLSCAASGFTIGSGWIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 466 aa, synthetic construct
MGWSLILLFLVAVATRVASEVQLVESGGGLVQPGGSLRLSCAASGFTIGSGWIHWVRQAPGKGLEWVAGIGPYGGYTYYADSVKGRFTISADTSKNTAYLQMNSLRAEDTAVYYCARFNDHWDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 468 aa, synthetic construct
MGWSLILLFLVAVATRVASEVQLVESGGGLVQPGGSLRLSCAASGFTIDDGFIHWVRQAPGKGLEWVAAIKPRQGDTSYADSVKGRFTISADTSKNTAYLQMNSLRAEDTAVYYCARFNDHWDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 466 aa, synthetic construct
MGWSLILLFLVAVATRVASEVQLVESGGGLVQPGGSLRLSCAASGFTIDDGFIHWVRQAPGKGLEWVAAIKPRQGDTSYADSVKGRFTISADTSKNTAYLQMNSLRAEDTAVYYCARFNDHWDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 468 aa, synthetic construct
MGWSLILLFLVAVATRVASEVQLVESGGGLVQPGGSLRLSCAASGFTIDDGFIHWVRQAPGKGLEWVAAIPPLQGKTYYADSVKGRFTISADTSKNTAYLQMNSLRAEDTAVYYCARFNDHWDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 466 aa, synthetic construct
MGWSLILLFLVAVATRVASEVQLVESGGGLVQPGGSLRLSCAASGFTIDDGFIHWVRQAPGKGLEWVAAIPPLQGKTYYADSVKGRFTISADTSKNTAYLQMNSLRAEDTAVYYCARFNDHWDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 468 aa, synthetic construct
MGWSLILLFLVAVATRVASEVQLVESGGGLVQPGGSLRLSCAASGFTIDDGFIHWVRQAPGKGLEWVATISPNTGHTNYADSVKGRFTISADTSKNTAYLQMNSLRAEDTAVYYCARFNDHWDLWMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 466 aa, synthetic construct
MGWSLILLFLVAVATRVASEVQLVESGGGLVQPGGSLRLSCAASGFTIDDGFIHWVRQAPGKGLEWVATISPNTGHTNYADSVKGRFTISADTSKNTAYLQMNSLRAEDTAVYYCARFNDHWDLWMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 237 aa, synthetic construct
MRVPAQLLGLLLLWLPGARCDGTDIQMTQSPSSLSASVGDRVTITCRASQDVYGYVAWYQQKPGKAPKLLISYTPGLYSGVPSRFSGSGSGTDFTLTISSLQPEDFATYYCQQYSRMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 468 aa, synthetic construct; identical to SEQ ID NO: 69

SEQ ID NO: 97 - protein, 466 aa, synthetic construct; identical to SEQ ID NO: 70

SEQ ID NO: 98 - DNA, 714 bp, synthetic construct
  source 1..714: mol_type = other DNA
atgagggtccccgctcagctcctggggctcctgctgctctggctcccaggtgcacgatgtgatggtaccgatattcaaatgacccagagcccgagcagcctgagcgcgagcgtgggagatcgcgtgaccatcacctgccgtgcaagccaggatgtttacgggtatgtcgcatggtatcagcagaaaccaggcaaagcgccgaaacttctgatatcttacacccctgggctgtatagcggcgtgccgtcgcgtttttcgggcagtggcagcggcacggactttaccctgacgatatcttccttacaaccggaggattttgcgacctactactgtcaacagtattatgatttcccgataaccttcggtcaaggcaccaaagtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 99 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgacgatgggttcattcattgggtgcgtcaagctcccggcaaggggctggagtgggtcgcggggattgggccctacgggggttacacatactatgccgacagcgtgaaaggtcgctttacgattagtgcggacaccagcaaaaataccgcgtacctgcagatgaatagcctgcgtgcggaagacacagcggtgtattattgcgcgcgtttcaacgaccactgggacctttggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 100 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgacgatgggttcattcattgggtgcgtcaagctcccggcaaggggctggagtgggtcgcggggattgggccctacgggggttacacatactatgccgacagcgtgaaaggtcgctttacgattagtgcggacaccagcaaaaataccgcgtacctgcagatgaatagcctgcgtgcggaagacacagcggtgtattattgcgcgcgtttcaacgaccactgggacctttggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 101 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcagggatgataagccgcagatgatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 102 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcagggatgataagccgcagatgatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 103 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttccgtgatacgcagtcgcagacgatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 104 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttccgtgatacgcagtcgcagacgatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 105 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcggtcatcctccgtatgattatatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 106 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcggtcatcctccgtatgattatatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 107 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttccagaatagtccgactaagactatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 108 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttccagaatagtccgactaagactatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 109 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcgctactcatgatcagcctctgatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 110 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcgctactcatgatcagcctctgatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 111 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattcagaatgcgtggattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgagatctgtggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 112 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattcagaatgcgtggattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgagatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 113 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattcatggttcttggattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 114 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattcatggttcttggattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 115 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccatttcgagttcgtatattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 116 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccatttcgagttcgtatattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 117 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgggtcgggttggattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 118 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgggtcgggttggattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 119 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcggctattaagccccgtcagggtgatacatcgtatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 120 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcggctattaagccccgtcagggtgatacatcgtatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 121 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcggctattcctcccctgcagggtaagacatattatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 122 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcggctattcctcccctgcagggtaagacatattatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 123 - DNA, 1487 bp, synthetic construct
  source 1..1487: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgactatttcgcccaatactggtcatacaaattatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 124 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgactatttcgcccaatactggtcatacaaattatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga

SEQ ID NO: 125 - DNA, 714 bp, synthetic construct
  source 1..714: mol_type = other DNA
atgagggtccccgctcagctcctggggctcctgctgctctggctcccaggtgcacgatgtgatggtaccgatattcaaatgacccagagcccgagcagcctgagcgcgagcgtgggagatcgcgtgaccattacctgccgtgcgagccaggatgtttatggctacgtcgcatggtatcagcagaaaccaggcaaagcgccgaaacttctgataagctacactccaggcctgtatagcggcgtgccgtcgcgtttttcgggcagtggcagcggcacggactttaccctgacgatatcttccttacaaccggaggattttgcgacctactactgtcaacagtattctaggatgccgattaccttcggtcaaggcaccaaagtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 126 - DNA, 1410 bp, synthetic construct
  source 1..1410: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggactacaagtgcaaggtctccaacaaagccctcccagcccccatcgagaaaaccatctccaaagccaaagggcagccccgagaaccacaggtgtacaccctgcccccatcccgggatgagctgaccaggaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 127 - DNA, 1401 bp, synthetic construct
  source 1..1401: mol_type = other DNA
atgggttggagcctcatcttgctcttccttgtcgctgttgctacgcgtgtcgcatctgaagtgcagctggtggaatcgggaggcggtctggtgcaacctggcggcagccttcgtctgagctgtgcggcgagcgggttcaccattgatgacggctttattcattgggtgcgtcaagctcccggcaaggggctggagtgggtcgcgggcattggcccctacggcggttatacatactatgccgacagcgtgaaaggtcgctttacgattagtgcggacaccagcaaaaataccgcgtacctgcagatgaatagcctgcgtgcggaagacacagcggtgtattattgcgcgcgtttcaatgatcattgggatctgtggatggattattgggggcagggcacccttgttaccgtgagctcggctagcaccaagggacccagcgtgttccctctggccccttgctctagatccacctccgagtccacagctgctctgggatgtctggtgaaggactacttccccgagcccgtgacagtcagctggaacagcggcgctctgacatccggagtgcacacattccccgccgtgctgcagtccagcggactgtactctctgagcagcgtggtgacagtcccctcctcctctctgggaaccaagacctacacatgcaacgtcgaccacaagccctccaacaccaaggtggacaaaagggtggagtccaagtatggccccccttgccccccttgtcccgcccccgagttcctcggaggcccctccgtgtttctgttccctcctaagcccaaggacacactgatgatctctagaacccccgaggtgacatgcgtggtggtggacgtgagccaagaggaccccgaggtccagttcaattggtatgtggacggcgtggaagtgcacaacgccaaaaccaagcctagagaggagcagttcaactccacctatagagtggtgtccgtgctcaccgtgctgcatcaagactggctcaacggcaaggagtacaagtgcaaggtctccaacaagggactgcccagcagcatcgagaagaccatcagcaaggccaagggacagcctagagaacctcaagtgtacacactgcccccttcccaagaggagatgaccaagaaccaagtgtctctgacatgtctggtgaaaggcttctaccctagcgacatcgccgtcgagtgggaaagcaacggccagcccgagaacaactacaaaacaacaccccccgtgctggactccgacggcagcttctttctgtactctagactgaccgtggacaagtctagatggcaagagggcaatgtgtttagctgcagcgtgatgcacgaggctctgcacaaccactacacccagaagtctctgtctctgtctctgggcaagtga